(19) United States
(12) Patent Application Publication
(10) Pub. No.: US 2025/0319596 A1
(43) Pub. Date: Oct. 16, 2025

(54) METHOD AND SYSTEM FOR ELECTROMECHANICAL SAFETY FOR ROBOTIC MANIPULATORS

(71) Applicant: Aescape, Inc., New York, NY (US)

(72) Inventors: Adam Johannes Nilsson, Jersey City, NJ (US); Tess C. Carella, Jersey City, NJ (US); Patrick C. Devine, Sayville, NY (US); Nicholas Akiona, New York, NY (US); Eric A. Litman, Brooklyn, NY (US); David N. Walsh, Brooklyn, NY (US)

(21) Appl. No.: 19/051,062

(22) Filed: Feb. 11, 2025

Related U.S. Application Data
(63) Continuation-in-part of application No. 17/959,777, filed on Oct. 4, 2022, now Pat. No. 12,397,430.
(60) Provisional application No. 63/553,027, filed on Feb. 13, 2024.

(51) Int. Cl. B25J 9/16; B25J 11/00; B25J 19/06

(52) U.S. Cl. CPC B25J 9/1674; B25J 11/008; B25J 19/06

(57) ABSTRACT

A robotic system includes a robotic arm that interacts with an object during operation. It further includes a locking component that, during operation, locks a multi-position component in an operating position. Responsive to a triggering event, locking of the multi-position component in the operating position is released and the multi-position component moves away from its operating position, facilitating separation between the robotic arm and the object.

### CROSS REFERENCE TO OTHER APPLICATIONS

This application claims priority to U.S. Provisional Patent Application No. 63/553,027 entitled ROBOTIC MANIPULATOR DAMPENING FOR ELECTROMECHANICAL SAFETY filed Feb. 13, 2024 which is incorporated herein by reference for all purposes.

This application is a continuation in part of pending U.S. patent application Ser. No. 17/959,777 entitled METHOD AND SYSTEM FOR ELECTROMECHANICAL SAFETY FOR ROBOTIC MANIPULATORS filed Oct. 4, 2022, which is incorporated herein by reference for all purposes.

### BACKGROUND OF THE INVENTION

Robotics have been used for various applications, including assembly manufacturing and device testing. For robotics used in manufacturing, various organizations, e.g., Occupational Safety and Health Administration (OSHA), have developed safety guidelines that focus on the “work envelope” (e.g., immediate vicinity or close proximity) of action of the robot, recommending that persons do not enter that work envelope or area of the robot when the robot is powered “on” or active. There are different types of work envelopes identified for robots, including, for example, maximum, restricted, and operating envelopes. These work envelopes can encompass both lateral and vertical areas of movement by the robot. The maximum work envelope space is the maximum area in which the moving parts of the robot can move. The restricted work envelope is a portion of the maximum work envelope which includes restrictions of the device, such as reach limitations, that establish an area not to be exceeded by the robot. The operating work envelope is a portion of the restricted work envelope that is utilized during normal performance by the robot.

A variety of types of accidents exist that can and have happened in use with industrial robots. For example, improper software programming has caused an operator working on the software to be struck by the associated robot. As another example, an operator inappropriately entering the robot's working envelope during operation was pinned by the robot. As another example, an operator accidentally turned on a robot while it was being serviced, causing the robot to strike the maintenance worker. Accordingly, several types of accidents can occur before, during and after use of the robot.

### BRIEF DESCRIPTION OF THE DRAWINGS

Various embodiments of the invention are disclosed in the following detailed description and the accompanying drawings.

FIG. 1 shows a diagram of a robot with a robotic arm, according to a first embodiment.

FIG. 2 shows a diagram of a robot with a robotic arm, according to a second embodiment.

FIG. 3 shows a block diagram associated with a robot, according to an embodiment.

FIG. 4A shows a diagram of a robot including two robotic arms, according to an embodiment.

FIG. 4B shows a section view of a joint with ratcheting functionality, according to an embodiment.

FIG. 4C shows a side view of the joint from FIG. 4B, according to an embodiment.

FIG. 4D shows a section view of a joint with ratcheting functionality, according to an embodiment.

FIG. 4E shows a side view of the joint from FIG. 4D, according to an embodiment.

FIG. 5 shows a diagram of a robot and a cushion, according to an embodiment.

FIG. 6A shows a diagram of a robot with an adjustable base, according to an embodiment.

FIG. 6B shows a block diagram visualizing a robot arm before tilting/moving/angling away from a base, according to an embodiment.

FIG. 6C shows a block diagram visualizing a robot arm after tilting/moving/angling away from the base, according to an embodiment.

FIG. 6D shows a zoomed in image for a joint area that visualizes a robot arm tilting/moving/angling away from a base, according to an embodiment.

FIG. 6E show a zoomed in image for a joint area when the robot arm is not titled/moved/angled away from a base, according to an embodiment.

FIG. 6F show a zoomed in image for a joint area when the robot arm is not titled/moved/angled away from a base, according to an embodiment.

FIG. 7 shows a diagram of a robot with a barricade, according to an embodiment.

FIG. 8 shows a diagram of a remotely-controllable robot, according to an embodiment.

FIG. 9 shows a flowchart of a method that can be performed by a robot, according to an embodiment.

FIG. 10A illustrates an embodiment of a robot base assembly.

FIG. 10B illustrates an embodiment of a robot base assembly.

FIG. 10C illustrates an embodiment of a robot base assembly in a robotic massage system.

FIG. 11 illustrates an embodiment of a robot-object separation system using a prismatic joint with guiderail.

FIG. 12 illustrates an embodiment of a robot-object separation system using a linkage-based component.

FIG. 13 illustrates an embodiment of a mass-based displacement system.

FIGS. 14A and 14B illustrate an embodiment of a robot-object separation. system.

FIG. 15 is a flow diagram illustrating an embodiment of a process for facilitating separation between a robotic arm and an object.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

The present disclosure relates to systems, methods, and apparatuses for electromechanical safety for robotic manipulators. More specifically, some embodiments of the present disclosure relate to a system, method, and apparatus for electromechanical safety for a robotic manipulator used for applying force to a deformable body.

The various embodiments described and illustrated herein are for the purpose of showing some example embodiments, and are not intended to limit in any way the scope.

In an embodiment, an apparatus includes a base and a robotic arm operatively coupled to the base via a connector. The robotic arm includes a set of links (i.e., rigid members or segments) interconnected by a set of joints. A first link from the set of links is operatively coupled to the connector. Each joint from the set of joints includes a brake from a set of brakes. The set of brakes includes a first subset of brakes and a second subset of brakes. Each brake from the set of brakes is configured to be enabled or disabled. The apparatus also includes an end effector (e.g., a tool) that is optionally positioned at an end of a sequence or arrangement of links and joints. The end effector is operatively coupled to the robotic arm via a second link from the set of links different from the first link. The apparatus also includes a controller, communicably coupled to at least one of the base, the robotic arm, or the end effector. The controller is configured to cause the robotic arm to perform a task. The controller is further configured to determine, during the task, that movement of the robotic arm is to be restricted. The controller is further configured to enable the first subset of brakes in response to determining that movement of the robotic arm is to be restricted. The controller is further configured to disable the second subset of brakes in response to determining that movement of the robotic arm is to be restricted.

In an embodiment, an apparatus includes a base and a robotic arm operatively coupled to the base via a connector. The robotic arm includes a set of links interconnected by a set of joints. A first link from the set of links is operatively coupled to the connector. Each joint from the set of joints includes a brake from a set of brakes, each brake from the set of brakes configured to be enabled or disabled. The apparatus also includes an end effector operatively coupled to the robotic arm via a second link from the set of links different from the first link. The apparatus also includes a controller, communicably coupled to at least one of the base, the robotic arm, or the end effector. The controller is configured to cause the robotic arm to perform a task, and to determine, during the task, that movement of the robotic arm is to be restricted.

In an embodiment, a non-transitory, processor-readable medium stores code representing instructions executable by a processor. The code includes code to cause the processor to cause a robot to perform a task that includes causing an end effector included in the robot to contact an object. The robot includes a set of links interconnected by a plurality of joints. Each joint from the plurality of joints includes a brake from a plurality of brakes. Each brake from the plurality of brakes is configured to be enabled or disabled. The plurality of brakes include a first set of brakes and a second set of brakes. The end effector is coupled to at least one of a link from the set of links or an attachment device coupled to the link from the set of links. The code also includes code to cause the processor to determine, during the task, that movement of the robot is to be restricted. The code also includes code to cause the processor to enable the first set of brakes in response to determining that movement of the robot is to be restricted. The code also includes code to cause the processor to disable the second set of brakes in response to determining that movement of the robot is to be restricted.

Robots used in industrial work have caused injuries due to a variety of errors. For example, such errors have included human error, control software error, mechanical failure, environmental interference, and unexpected energy surges—all of which could lead to a change in a robot's intended performance. Such potential errors should be accounted for to ensure the safety of those working with or near such robots. Moreover, when the use of robots involves direct contact with a human or other entity, the safety of those being manipulated by the robots should also be accounted for with redundant safety measures.

Gates and/or cages have been recommended for industrial robots, when possible, to prevent persons from accidentally entering the area of action of the robot. However, in the case where a robot is being used by a human operator in close proximity to the robot, or where a robot is in physical contact with a subject entity, e.g., a human, a gate or cage may not always be a viable solution. Similarly, other proposed safety devices such as floor sensors, motion sensors or light curtains designed to stop a robot whenever a person approaches or enters the work envelope of the robot may not always be viable solutions.

The safety implications of a human being worked on by a robot, and/or of a robot operating with an operator in close proximity thereto, are complex and not adequately addressed by known systems. For example, various robot arms available in the marketplace use joint brakes that either unlock when there is a loss of power (which could cause the robot to fall on the subject human/entity and/or operator), or lock when there is a loss of power, causing the robot to become rigid and immovable, and possibly trapping the subject human/entity and/or operator. As mentioned above, floor sensors, and motion sensors, and light curtains do not always provide useful protection when a subject human and/or operator is in close proximity to or contact with a robot. For robots that act on humans more directly or invasively, such as robots used in medical surgery, published OSHA guidelines do not appear to adequately protect the subject human or object that the robot acts upon, and/or the operator of the robot, all of whom are in the work envelope while the robot is active. Accordingly, there exists a need for safety features, in a robot system configured to act upon a person/soft or deformable body/object, that appropriately protect all entities located in the vicinity of the robot system, and that do not unnecessarily cause harm or damage to the robot system during power down or disablement. Moreover, especially in the area of medical or massage applications, improved safety features of a robot system are desirable for better protecting the person/operator controlling and/or working near the robot, and for better protecting the person/entity that the robot is working on during a procedure.

Systems and methods set forth herein address the shortcomings of known robot systems discussed above. More specifically, one or more embodiments of the present disclosure provide for safety features including method, system, and apparatus embodiments, for a robot system acting on an object, such as a human, body, deformable object, and/or the like. The safety features can include a configuration in which a person (e.g., a human being acted on/contacted by a robot) can control the robot remotely, optionally including the ability to remotely power down or disable the robot. Alternatively or in addition, the safety features can include the ability to power down or disable (e.g., remotely and/or automatically) an autonomously working robot acting upon a person/entity. Alternatively or in addition, the safety features can include a configuration in which a person being acted upon by the robot can power down or disable the robot.

One or more method, system, and apparatus embodiments of the present disclosure provide for safety features for a robot system acting in close proximity to a human/body/object (e.g., within 1 inch, within 2 inches, within 6 inches, within 1 foot, within 5 feet, within 10 feet, etc. of the human/body/object).

One or more embodiments of the present disclosure provide for a system in which computer readable instructions are provided, which can be stored on a memory medium, and which can be executed by a controller (e.g., a processor) to disable and/or power down a robot system in a safe manner for any human or body being acted upon at that time.

One or more embodiments of the present disclosure provide for safety features to enhance the safety features currently provided with an “off the shelf” robot from a manufacturer. For example, an “off the shelf” robot may include the capacity for an e-stop or electronic shutdown. If a robot is powered down electronically, however, that may automatically lock the joints of the robot such that the joints cease to move or cannot be moved from a current position. This can effectively trap and/or hurt a human or soft body being operated on by the robot.

One or more embodiments of the present disclosure provide for a robot having at least one robotic arm (or “robot arm”), the robot arm being comprised of an interconnected set of links and powered joints. The robot arm includes manipulators which support or move the wrist of the robot and the end effector. The end effector can be a specialized touch point or other end effector touchpoint designed for attachment to the robot wrist and designed to perform the intended task of making contact with a person or soft body or object.

One or more embodiments of the present disclosure provide for a robot having at least one method for disablement, providing redundancy and improving fault tolerance. The robot arm is configured to act upon an entity such as a person, an animal, a soft body, or an object, whether standing, sitting, or lying down. If lying and/or sitting down, the entity can be disposed on a firm support structure such as a table, chair, or other support structure. The robot actions are controlled by a controller (e.g., a processor), the controller providing electronic instructions to the robot arm to make contact between the touch point and the entity, and to effect one or more actions. The actions can be, among other things, e.g., a massage, a treatment of a specific area of the entity, and/or resistance testing of a specific area of the entity.

One or more embodiments of the present disclosure provide for an electronic shut off of the robot. The electronic shut off can be implemented in a variety of ways such as, for example, as a disabling switch or button on the robot itself, as a separate remote switch (whether wired or wireless (e.g., Bluetooth® enabled)), via a device software application (“app), and via a command issued by the robot system controller.

One or more embodiments of the present disclosure provide for one or more disabling features of the robot, which can be used in conjunction with or triggered by a shutdown of the robot. Examples of such disabling features can include functionality for disabling certain movements, turning power off, enabling a subset of brakes, disabling a subset of brakes, and/or the like.

One or more embodiments of the present disclosure provide for a disabling feature of the robot in which a mechanical or electro-mechanical brake of one or more of the brakes associated with the joints of the robot arm is removed or otherwise disabled (i.e., not locked/not braked) so that in the event of a shutdown of the robot, the robot arm is not locked in place and instead can be manipulated manually. For example, in the event that a robot is acting upon a person having a massage, and the robot is powered down either intentionally or unintentionally, one or more of the joints of the robot arm are not braked or are not locked. This can allow the person having the massage or a person nearby to manually move the robot arm away from the body to allow the person to leave the treatment area.

One or more embodiments of the present disclosure provide for a disabling feature of the robot in which a mechanical or electro-mechanical brake of the wrist of the robot is removed or disabled such that it cannot be enabled/re-enabled, e.g., automatically and/or in response to a reduction, fluctuation, or loss in power. Similarly, one or more embodiments of the present disclosure provide for a disabling feature of the robot in which a mechanical or electro-mechanical brake of a robot (e.g., t-axis robot) is removed or disabled such that it cannot be enabled/re-enabled. For example, for an n-joint robot arm (where n can be any number, such as 4, 5, 6, 7, 8, 9, 10, etc.), then the (n−1)th and/or nth joint brake (where the joint closest to the base is the 1st joint and the joint at the other end of the robot arm closest to the end effector is the nth joint) may be removed/disabled so that it cannot be enabled if the robot loses power or acts on an instruction to stop function, et al. As one example, if there are 6 joints, then the 5th and/or 6th joint brake may be removed/disabled so that it cannot be enabled. As another example, if there are 7 joints, the 6th and/or the 7th joint brake is removed so that it cannot be enabled. Of course, other brakes can likewise be removed/disabled, such as the (n−2) brake, (n−3) brake, and/or the like. In some implementations, the mechanical or electro-mechanical brake of the wrist of the robot may be disabled electronically. Alternatively or in addition, the robot may include one or more physical mechanisms (e.g., actuators) that can be adjusted during operation of the robot to prevent or permit the mechanical or electro-mechanical brake of the robot to engage. Alternatively, a mechanical or electro-mechanical brake (e.g., of the wrist or other joint) of the robot may be physically removed from the robot during manufacture of the robot arm.

One or more embodiments of the present disclosure provide for an override of at least one brake associated with a joint of the robot manipulator or arm. In some implementations, an override of the at least one brake is effected by at least one of a safety-rated force sensor, contact sensor, or soft/hard button. In an embodiment, there is an override of the nth and/or (n−1)th brake of a robot arm, using at least one of a safety-rated force sensor, a contact sensor, and/or a soft/hard button.

One or more embodiments of the present disclosure provide for a robot arm or manipulator to be disabled at one or more joint brakes in the event of a dynamic “stop” function by a user, an operator, an operational error, and/or a loss of functionality or power. For example, upon a stop-function of the robot arm or manipulator (“robot”), all of the currently disengaged brakes that were not disabled would engage to lock their respective robot joints. For those joint brakes that were removed prior to this stop-function and/or that received a command from a controller (e.g., a software controller) to disable or not-lock at least one joint brake, a user or operator can then manually move, manipulate and/or push the robot away from an object or body to allow for removal/departure of the object or body. In some implementations, the disabling of one or more joint brakes, but not of all joint brakes, prevents the robot from falling on or otherwise damaging or impairing the object or body or operator using or engaging with the robot. In some implementations, there is a disabling and/or removal of one or more joint brakes of the joints that allow only for lateral movement of the robot in the event of a stop-function, operational error, power loss, or other similar situation. In some implementations, there is a disabling and/or removal of at least one lateral movement joint brake and one upward vertical movement joint brake, to better allow for the manual movement of the robot away from, e.g., a user positioned on a massage table who was previously being massaged by the robot.

In some implementations, by not disabling and/or removing every joint brake, the robot stays intact and does not get damaged unnecessarily (e.g., from falling) in the event of a stop-function, operational error, power loss, or other situation.

One or more embodiments of the present disclosure provide for a robot arm or manipulator to fall back with (e.g., tilt away from) its base attachment in response to an emergency stop condition being triggered, such as a loss of power, a user input, some other safety threshold, and/or the like. One or more embodiments provide for a solenoid located in a portion of the base of the robot or other attachment location, so that when the electric current ceases, the effective magnet behavior of the solenoid ceases, and the portion having the solenoid as an attachment ceases to be attached and lifts up or disengages, allowing the robot arm or manipulator to move away from the entity in a safe manner. One or more embodiments provide for a solenoid at one portion of the robot base attachment to a support or table or standalone base, and for a regular attachment apparatus of a screwed hinge, spring attachment, or other attachment which allows the robot arm or manipulator to angle back or tilt or otherwise relocate away from the entity. One or more embodiments include (A) a solenoid positioned in a base of the robot and/or robot arm, the base being either attached to a supported structure such as a table, or standalone, and (B) an attachment mechanism such as a screwed hinge, spring, etc., involved in facilitating an angling, tilting, or other repositioning of the robot arm/manipulator to move the robot arm/manipulator away from the entity in response to a stop condition. In some implementations, the solenoid can keep the robot stable and engaged during operation, but not after disengaging (e.g., from a stop condition). Although one or more implementations herein are described using a solenoid, other electronically controlled brakes can be used additionally or alternatively. Although the foregoing describes implementations in which a solenoid is part of the robot base attachment to a support or table or standalone base, one or more solenoids can alternatively or additionally be positioned at or in an end effector flange of the robot.

One or more embodiments of the present disclosure provide for a mechanical pressure switch or button that disengages at least one of the brakes associated with one or more of the joints of the robot manipulator or arm. One or more embodiments provide for a mechanical pressure switch or button that disengages at least one brake associated with a last and/or second to last joint or link or wrist joint of the robot. One or more embodiments provide for a mechanical pressure switch or button that disengages at least one brake associated with at least one joint or link or wrist joint of the robot.

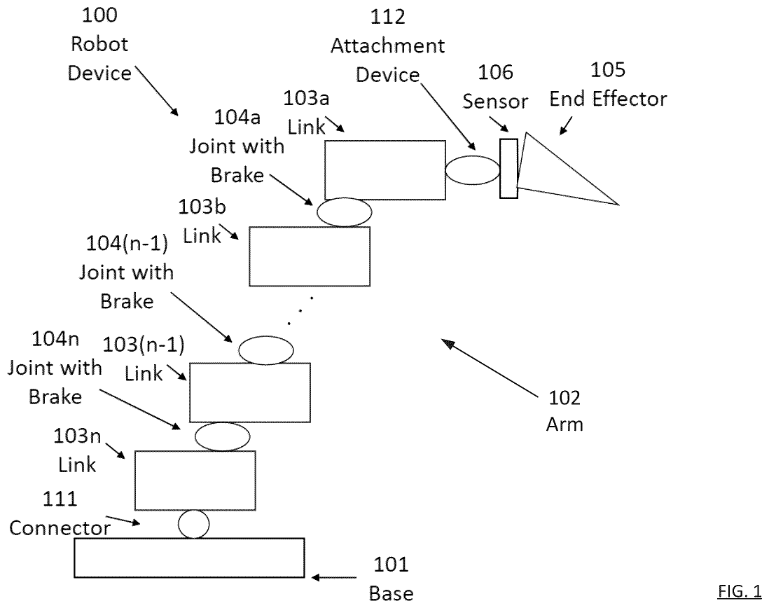

FIG. 1 shows an example embodiment of a robot device 100. The robot device 100 has a fixed base 101. The fixed base 101 can be fixed or permanently attached or removably attached to a base structure, support structure, massage table, floor, wall, ceiling, movable carriage, or other structure. The fixed base 101 can be attached to a rail system or block or other structure movably attached to a rail system, allowing the robot device 100 to be moved along the side of a table, chair, wall, floor, or other structure. The robot device has an arm 102 which is pivotably connected, via connector 111, to the fixed base 101. The arm 102 includes one or more segments or links 103a . . . 103n (where n can be any number, such as 2, 3, 4, 5, 6, 7, 8, 9, 10, etc.). Each of the links 103a . . . 103n are connected (e.g., interconnected) to each other at a joint portion 104a . . . 104n. At an end of the link 103a, an end effector or touch point 105 can be removably attached (e.g., via a device 112). The device 112 can be a joint, screw, magnet, hinge, adhesive, or other available attachment device. In an embodiment, at least one of a sensor, a force sensor or a detection device 106 is located at, on, or near at least one of the device 112, the end effector 105, or the link 103a. In an embodiment, the end effector 105 is attached directly to the link 103a (e.g., without device 112 and/or sensor 106). In an embodiment, the sensor 106 detects a force being exhibited by the robot device 100 on an entity (not shown in FIG. 1) that exceeds a threshold level and signals an immediate electronic shutdown of the robot device (e.g., automatically and without requiring human intervention). The threshold level can be stored in a memory (not shown in FIG. 1) (e.g., a local database, remote database) or other location that is accessible by a controller (not shown in FIG. 1) which operates the robot device 100 and/or gives instructions or commands to the robot device 100.

The controller (not shown in FIG. 1) can be, for example, a hardware based integrated circuit (IC) or any other suitable processing device configured to run and/or execute a set of instructions or code. For example, the controller can be a general-purpose processor, a central processing unit (CPU), an accelerated processing unit (APU), an application specific integrated circuit (ASIC), a field programmable gate array (FPGA), a programmable logic array (PLA), a complex programmable logic device (CPLD), a programmable logic controller (PLC) and/or the like. In some implementations, the controller can be configured to run any of the methods and/or portions of methods discussed herein. The controller can be housed at any one or more components of the robot device 100, or somewhere different than the robot device 100. Signals sent by the controller can be communicated to one or more components of the robotic device 100 (e.g., via a system bus), such as base 101, connector 111, joint portion 104a . . . 104n, links 103a . . . 103n, device 112, sensor 106, end effector 105, and/or a combination thereof. In some implementations, the controller continuously sends a signal (e.g., a power or command signal) and/or keeps a pin tied ‘high’ to prevent the brakes from engaging. The removal of the signal, in turn, can cause the brakes to engage.

The memory (not shown in FIG. 1) can be, for example, a random-access memory (RAM), a memory buffer, a hard drive, a read-only memory (ROM), an erasable programmable read-only memory (EPROM), and/or the like. The memory can be configured to store data used by the controller to perform the techniques discussed herein. In some instances, the memory can store, for example, one or more software programs and/or code that can include instructions to cause the controller to perform one or more processes, functions, and/or the like. In some embodiments, the memory can include extendible storage units that can be added and used incrementally. In some implementations, the memory can be a portable memory (for example, a flash drive, a portable hard disk, and/or the like) that can be operatively coupled to the controller. In some instances, the memory can be remotely operatively coupled with the robot device 100. For example, a remote database device (not shown in FIG. 1) can serve as a memory and be operatively coupled to the robot device 100. The memory is operatively coupled to the controller.

In an embodiment, the sensor 106 (e.g., a force sensor and/or a detection device) measures the force or other measurable matter by the end effector or touch point 105 in contact with an entity, and compares that measurement to a predetermined threshold level. If the measurement exceeds the threshold level, a remedial action can be triggered, such as effecting an alarm/buzzer or other light or sound notification. If the measurement exceeds the threshold level, this can trigger a command by the controller to lock, partially lock, stop, and/or move (e.g., to a predefined position), depending upon the preset controller command. The controller can optionally also trigger an electronic shutdown of the robot device 100.

In an embodiment, a user or operator can push a button (not shown in FIG. 1; or any other similar component) that immediately initiates a stop-function of the robot device 100 (e.g., arm 102). The button can be located anywhere on the robotic device 100, such as at/on the fixed base 101, the connector 111, joint portion(s) 104a . . . 104n, link(s) 103a . . . 103n, attachment device 112, sensor 106, end effector 105, and/or a combination thereof. Additionally or alternatively, the button can be located remote from the robotic device 100, but have wireless and/or wired communication capability with the robot device 100.

The end effector 105 can be any type of end effector 105, such as a gripper, a roller, a suction cup, a powered tool, a massage tool, and/or the like. In some implementations, the end effector is shaped for performing a massage technique, such as pinning, rolling, stretching, grabbing, and/or the like.

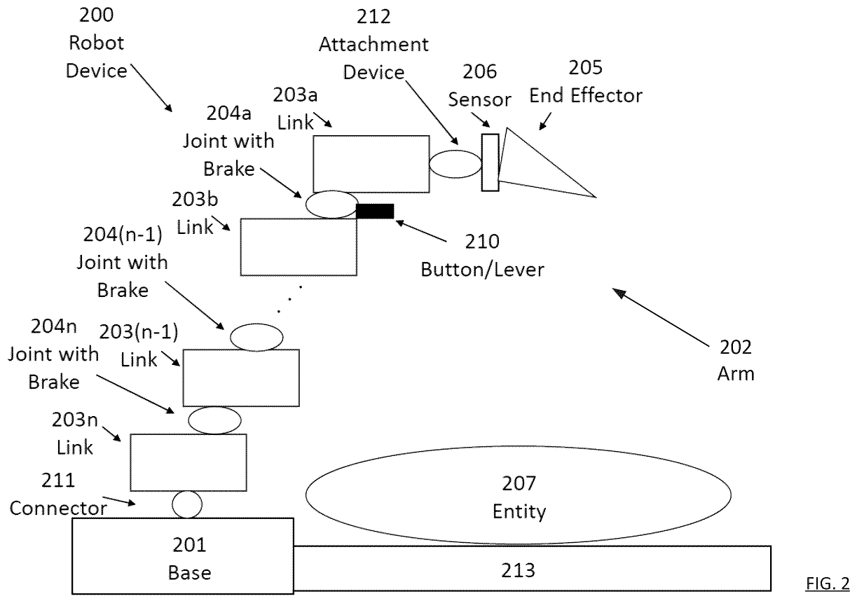

FIG. 2 shows an example embodiment of a robot device 200 acting upon an entity 207 such as an object, soft body, or human/animal body. Similar to robot device 100, robot device 200 can include a memory and a controller operatively coupled to the memory. The robot device 200 has a fixed base 201. In an embodiment, the fixed base 201 can be fixed or permanently attached or removably attached to a base structure 213 such as a support structure, massage table, floor, wall, ceiling, movable carriage, or other structure. In an embodiment, the fixed base 201 can be attached to a rail system or block or other structure movably attached to a rail system, allowing the robot device to be moved along the side of a table, chair, wall, floor, ceiling, housing, or other structure. The robot device 200 has an arm 202 which is at least one of connected, movably connection and pivotably connected, via connector 211, to the fixed base 201. The arm 202 includes one or more segments or links 203a . . . 203n (where n can be any number, such as 2, 3, 4, 5, 6, 7, 8, 9, 10, etc.). Each of the links 203a . . . 203n are connected to each other at a joint portion or joint portions with brake 204a . . . 204n. In an embodiment, at least one of joint portion 204(n−1) and 204n, the brake can be removed (e.g., mechanically removed) prior to use of the robot device 200. This can allow that, when a stop-function, malfunction, or loss of power occurs, an operator or user can move at least one or more portions of the robot arm 202 so that a user is not trapped by the robot arm 202. [0051] In an embodiment, an end effector or touch point 205 can be removably attached via a device 212 at the end of the robot arm 202 and at link 203a. The device 212 can be a joint, screw, magnet, hinge, adhesive, welding, or other available attachment device or method. In an embodiment, a force sensor or detection device 206 is located at, on, or near at least one of the attachment device 212, the end effector 205, or the link 203a. In an embodiment, the end effector 205 is attached directly to the link 203a (e.g., without device 212 and/or sensor 206). In an embodiment, the sensor 206 detects, e.g., a force being exhibited by the robot device 200 on an entity 207 that exceeds a threshold level and signals an immediate electronic shutdown of the robot device (e.g., automatically and without requiring human intervention). In an embodiment, the sensor 206 is an electromechanical sensor that triggers an electronic signal that shuts down the robot arm 200 in connection with the electromechanical sensor 206, upon sensing a specific force or improper direction of the end effector 205. In an embodiment, the sensor 206 is an electromechanical sensor 206 that is associated with a standalone controller and memory storage that compares the sensed data (e.g., force data) with at least one preset threshold, and effects at least one of a shutdown of the robot arm 202 and one or more robot arm joint brakes. Sensor 206 can be any type of sensor. In some implementations, arm 202 does not have to be an “arm,” but can have a different shape. The use of a robot arm in the embodiments described herein is meant for explanatory purposes and not intended to limit the scope of the invention.

In an embodiment, the entity 207 (or someone else different than the entity 207) can push a button or lever 210 that initiates immediately at least one of the at least one joint portion 204a . . . 204n to move at least one or more of the arm links 203a . . . 203n up or in a direction away from the entity 207. The button or lever 210 can be located at one or more of the joints to disable a brake of the joint.

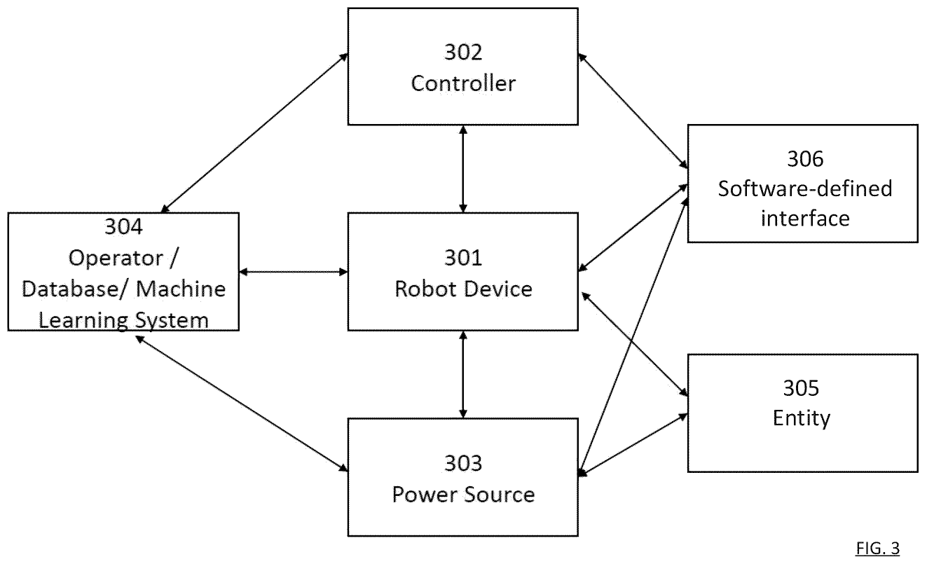

In FIG. 3, a robot system embodiment is shown having a robot 301 which is situated in order to act upon an entity 305, such as an object, soft body, or human. The robot 301 is comprised of a set of links that are interconnected by a set of powered joints, such as those shown in the embodiments of FIGS. 1 and/or 2. The robot 301 is powered by a power source 303 using AC or DC current, which may be, for example, a portable battery or otherwise. The robot 301 and the controller 302 communicate via direct connection, LAN, WLAN, Bluetooth, or other available connection. The controller 302 provides computer software commands to the robot 301 in order effect an action by the robot 301. The controller 302 can be a processor, computer, or a networked computer software system, or other available device. An additional entity 304—such as an operator or a database of instructions and commands, or a machine learning computer software system—can be present to give commands and/or control the controller 302. The entity 305 being acted upon by the robot 301 can have access to a software-defined interface (e.g., processor tablet, remote control, mobile device, or other computer software device) 306 capable of giving commands and/or requests to the controller 302, robot device 301, and/or power source 303. The entity 305 being acted upon by the robot device 301 can have access directly to the robot 301 and/or power source 303. This access by the entity 305 or software-defined interface 306 can allow, for example, for the entity 305 to stop the robot 301 in the event of malfunction and/or manipulate the robot 301 in the event of unexpected power loss or event.

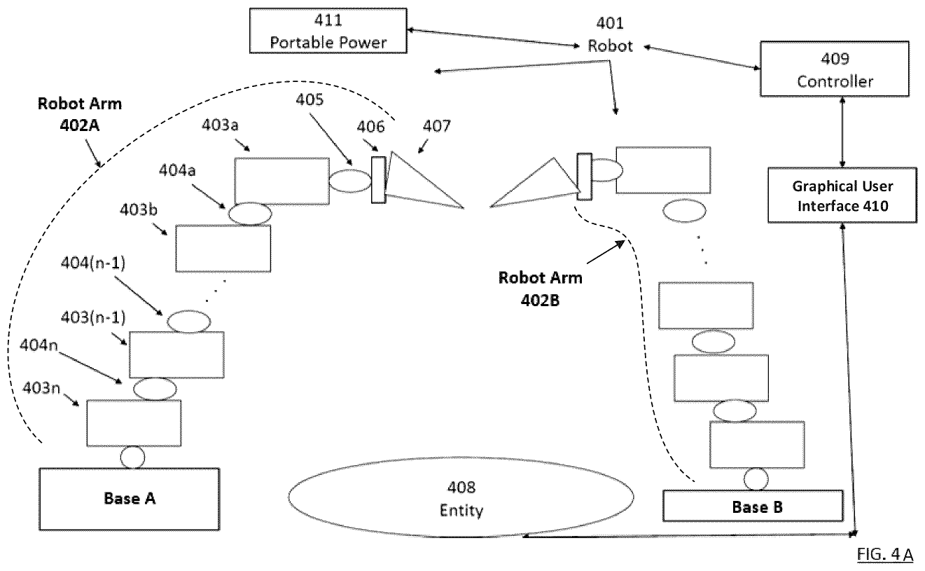

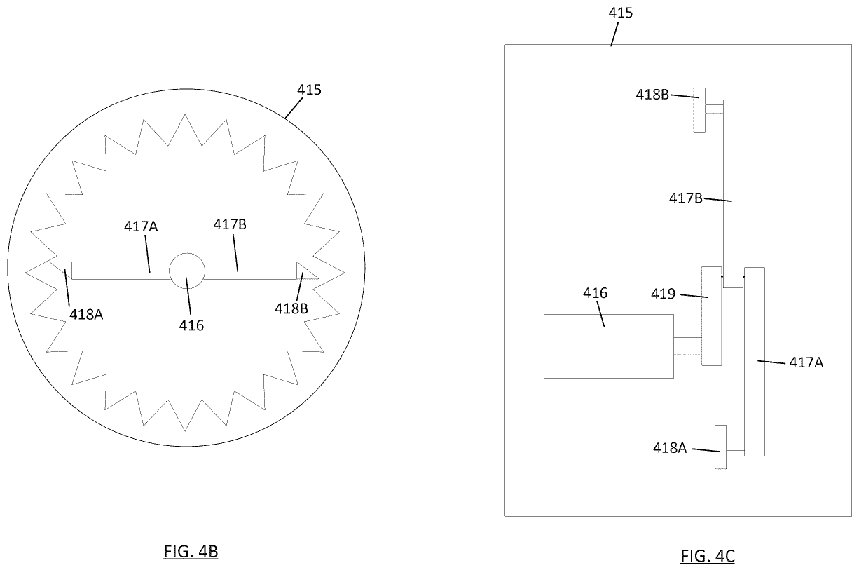

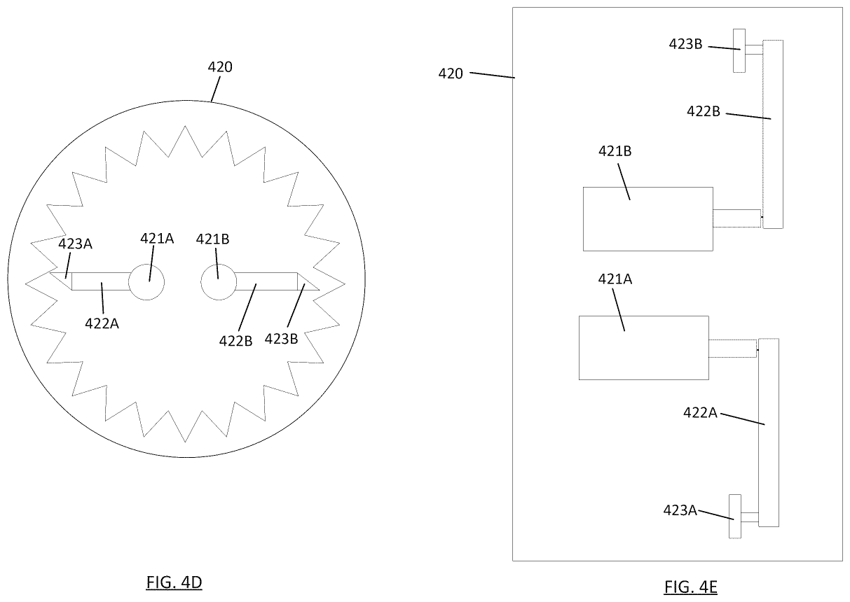

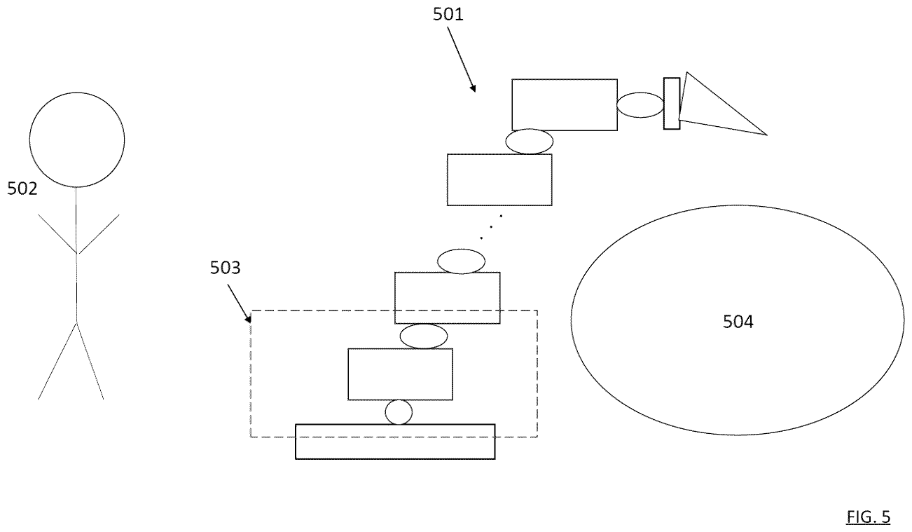

A system embodiment of a robot 401 having two robot arms (402A, 402B) is shown in FIG. 4A, with the robot arm 402A including a base “A,” an interconnected set of links 403a . . . n and powered joints 404a . . . n. The robot arm 402A includes a manipulator 405 that supports or moves a wrist 406 of the robot arm 402A and an end effector 407. The end effector 407 can be a specialized touch point or other end effector device designed for attachment to the robot wrist 406 and designed to perform an intended task that may include making physical contact with an entity 408. In some implementations, the entity 408 includes at least one of a human person or other soft body or object 408. In some implementations, the robot 401 is electronically controlled by a controller 409 that sends commands to one or more electronic components of the robot 401, e.g., to at least one actuator, in order to effect the commands. The controller 409 can be directly controlled by the entity 408 via a compute device (e.g., via a graphical user interface (GUI) 410 displayed at the compute device and/or via a software application or “app” running on the compute device) and/or can be controlled automatically by at least one of a processor, a programmable logic controller (PLC), a computer system, a networked computer system, or a machine learning computer software program/system (e.g., according to pre-programmed routines, rules, or other code), or can be controlled via a hybrid automatic-manual system. Robot arm 402B includes a base “B,” and can include some or all of the structure and/or functionality of robot arm 402A as described herein.

In some implementations, when the robot 401 does not execute a command properly, or another malfunction occurs, the robot 401 (e.g., via the controller 409) shuts down the robot, disables at least one feature of the robot 401, enables at least one feature of the robot 401, and/or modifies at least one feature of the robot 401. For example, in some implementations, when the robot 401 is disabled, the robot arm joints 404a . . . n connecting the different links 403a . . . n of the robot arm 402A are not powered. When this occurs, the robot arm joints do not allow movement, and are locked in place. Each of these joints are locked using a joint brake which can be electronic, electromechanical or mechanical. It can happen that when the robot arm 402A locks in place, the entity 408 may be trapped and/or enclosed by the robot arm 402A or trapped against or near a structure by the robot arm 402A. This can be a dangerous situation, for example, if a person getting a massage is trapped on a massage table by the robot arm. In this example, the person may be lying face down, and unable to escape or even see what is occurring. Further, the person may be unable to dislodge a mechanical brake(s) of one or more joints of the robot arm 402A. Accordingly, in this situation, an embodiment of a localized or portable power source 411 associated, attached, or in some way able to give limited power to the robot arm 402A is possible, allowing for the system to effect an automatic withdrawal of the robot arm 402A from the area of the entity 408. This allows the entity 408 to depart safely.

In some embodiments, the robot 401 is configured to automatically reposition one or more components thereof (e.g., the base “A,” the robot arm 402A (or any component or grouping of components thereof), the base “B,” and/or the robot arm 402B (or any component or grouping of components thereof)), to remove or reduce a force applied to the entity 408, for example in response to a loss of power, a power fluctuation, a reduction in power, or a detected abrupt movement meeting predefined criteria. The automatic repositioning can include one or more of: translating the one or more components along a direction extending away from the entity 408, rotating the one or more components away from the entity 408, reducing a force applied to the entity 408 by the one or more components, removing a force applied to the entity 408 by the one or more components, or causing the end effector to cease making contact with the entity 408. The automatic repositioning can include locking of one or more brakes associated with the one or more components, where the default during operation is an unlocked condition. Similarly, the repositioning can include unlocking of one or more brakes associated with the one or more components, where the default during operation is a locked condition (e.g., base “A” and/or base “B”).

In some embodiments, at least one of the robot arm 402A (i.e., any component(s)/joint(s) thereof) or the robot arm 402B (i.e., any component(s)/joint(s) thereof) includes a reconfigurable ratchet to selectively limit a first direction of movement thereof, and to facilitate incremental movement thereof in a second direction opposite the first direction. The ratchet can be a mechanical device that permits continuous or discontinuous (e.g., stepped) linear or rotational movement along only a first direction while preventing movement along a second direction opposite the first direction. The ratchet optionally includes a mechanical switch that is switchable between/among two or more positions, for example such that when the mechanical switch is in a first position, the ratchet permits movement in the first direction but not in the second direction, and when the mechanical switch is in a second position, the ratchet permits movement in the second direction but not in the first direction. In some implementations, the ratchet is configured to lock (i.e., to prevent movement in) in each of a first direction and a second direction. The ratchet can include a plurality of teeth configured to engage/mate with complementary-shaped cogs, teeth, or “pawls.” In some implementations, the default condition of the ratchet during operation of the robot arm 402A/402B is an unlocked condition, and the cogs, teeth, or pawls may be spring-loaded or otherwise mechanically retained in the unlocked position until engagement of the ratchet is triggered. In some implementations, movement of the robot arm 402A and/or robot arm 402B includes a mechanical and/or electronic limiter to prevent the robot 401 from tipping over or otherwise becoming imbalanced, while the ratchet is being used.

FIG. 4B shows a section view of a joint 415 that can perform a ratcheting function, according to an embodiment. As the motor 416 rotates, pawls 418A, 418B can each extend and/or retract (e.g., via a solenoid included in pawls 418A, 418B). For example, FIG. 4B shows a first configuration, where both pawls 418A, 418B are extended such that the joint 415 will not rotate substantially (e.g., will not rotate greater than a distance of one tooth spacing). In some implementations, the motor 416 can rotate such that, via links 417A, 417B and crank 419 (shown in FIG. 4C; not shown in FIG. 4B), both pawls 418A, 418B are retracted and joint 415 can rotate substantially freely in a clockwise and/or counterclockwise direction. In some implementations, the motor 416 can rotate such that pawl 418A is extended and pawl 418B is retracted so that the joint can rotate substantially in a first direction (e.g., clockwise), but not a second direction (e.g., counterclockwise). In some implementations, the motor 416 can rotate such that pawl 418A is retracted and pawl 418B is extended so that the joint cannot rotate substantially in the first direction (e.g., clockwise), but can rotate in the second direction (e.g., counterclockwise). In some implementations, the pawls 418A, 418B each include a solenoid that can extend or retract, thereby configuring the joint 415 to rotate substantially in the first direction but not the second direction, the second direction but not the first direction, both the first direction and the second direction, or neither the first direction nor the second direction. FIG. 4C shows a side view of the joint 415 from FIG. 4B, according to an embodiment. As the motor 416 rotates, via the crank 419 and links 417A, 417B, the pawls 418A, 418B can extend and/or retract.

FIG. 4D shows a section view of a joint 420 that can perform a ratcheting functionality, according to an embodiment. Compared to joint 415 from FIG. 4B, joint 420 includes two motors 421A, 421B. Pawl 423A can extend or retract via link 422A as motor 421A rotates, while pawl 423B can extend or retract via link 422B as motor 421B rotates. Through combinations of pawls 423A, 423B being extended or retracted, the joint 420 can be configured to rotate substantially in a first direction (e.g., clockwise) but not a second direction (e.g., counterclockwise), the second direction but not the first direction, both the first direction and the second direction, or neither the first direction nor the second direction. FIG. 4D shows a side view of a joint 420, according to an embodiment. As motor 421A rotates, pawl 423A can extend and/or retract via link 422A, while as motor 421B rotates, pawl 423B can extend and/or retract via link 422B.

Although each of joints 415, 420 is shown as including two pawls, in other implementations, more than two pawls can be used (e.g., for redundancy and/or additional functionality). Although the motors, pawl, links, cranks, and/or rotating gears of joints 415, 420 are shown as being located interior to joints 415, 420, in some implementations, the motors, pawls, links, cranks, and/or rotating gears can be located exterior to joints 415, 420.

In some embodiments, an additional joint is positioned at the base of the robot arm 402A and/or 402B and the base and/or the additional joint can be actuated or repositioned using a solenoid and/or a latch mechanism. In some such implementations, the actuation/repositioning of the additional joint is accomplished purely mechanically (i.e., not via software). For example, the additional joint can be configured for mechanical actuation using one or more of: spring-loading, gravity-based movement, rotational force (e.g., a spinning gear or motor), a locking mechanism (e.g., pins and slots), one or more solenoids configured to engage/disengage, multiple solenoids configured to engage/disengage, etc. Moreover, the additional joint can be configurable/“set” to any of variety of different positions and actuation behaviors. In other implementations, the actuation/repositioning of the additional joint is accomplished via software. Any joint described herein can include any combination of the functionalities set forth in this paragraph.

In some implementations, a safety device can be employed at and/or near a robot to protect an operator of the robot. For example, as shown in FIG. 5, to protect an operator 502 from the robot 501 acting in a non-normal way or acting under shutdown procedures, a safety device (in the form of a cushion 503) can be included in the environment of the robot 501. The robot 501 may have a normal performance area 504, but may sometimes depart from the normal performance area 504. In some implementations, the safety device is a cushion 503 that can either surround the base of the robot 501 or an upper portion of the robot 501. The cushion 503 can be made of a variety of materials, such as foam, feather, polyester, wool, leather, nylon, and/or the like. In some implementations, the cushion 503 can be an inflatable or partially inflatable cushion which deploys upon being triggered, for example in response to a power loss or malfunction of the robot 501 or other predefined event. For example, the cushion 503 can store air and inflates in a manner similar to that of compact air cushions used in motor vehicles when an impact is sensed. The cushion 503—whether already there to warn the operator 502 regarding work envelope area or whether inflated due to a sensed error—can prevent the operator 502 from getting unnecessarily close to the robot 501. The cushion 503 being inflatable effectively alerts the operator 502 of the sensed unexpected event, as well as provides a safe manner of contact.

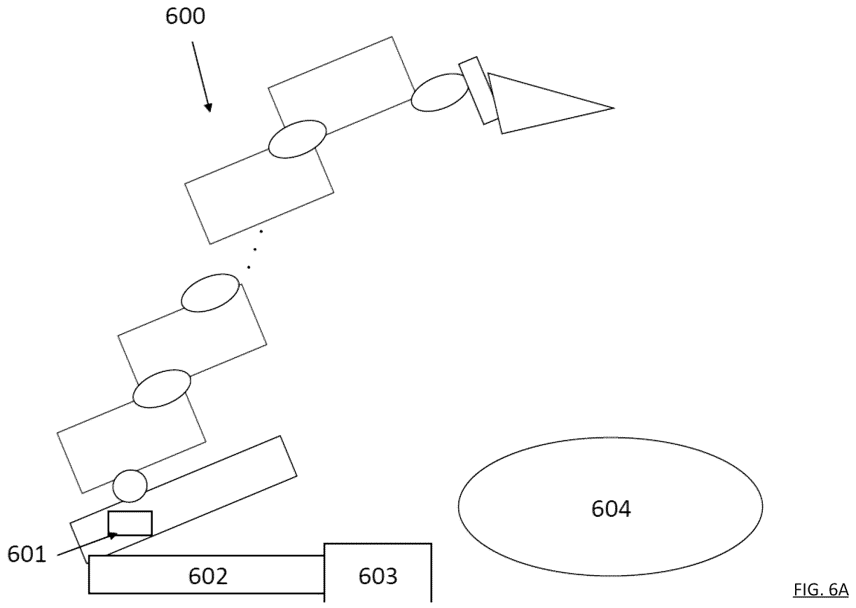

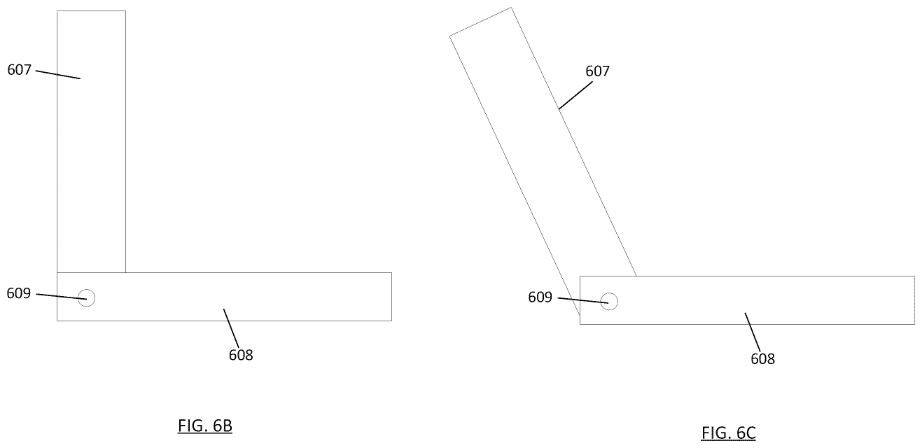

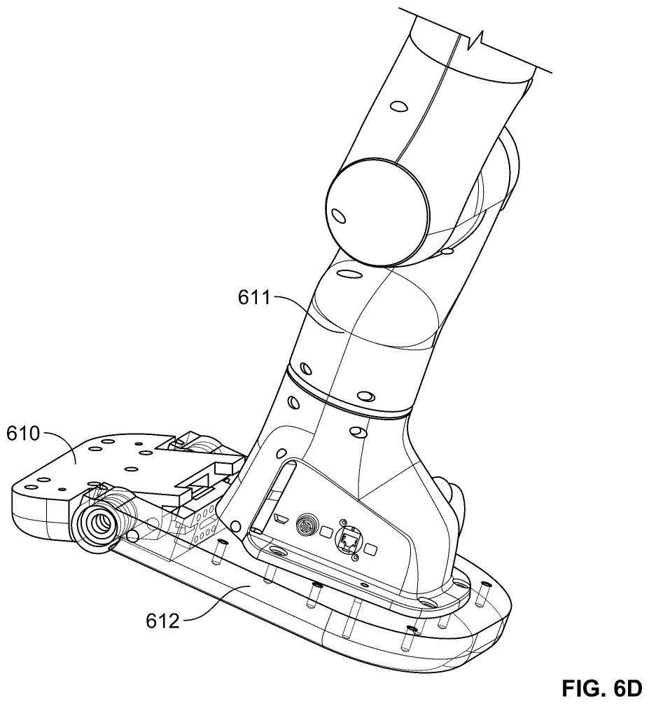

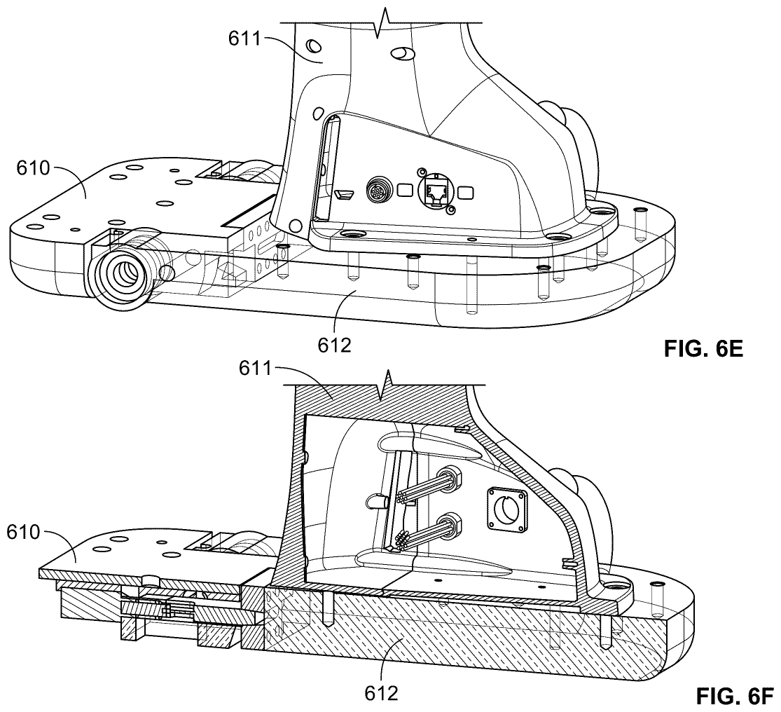

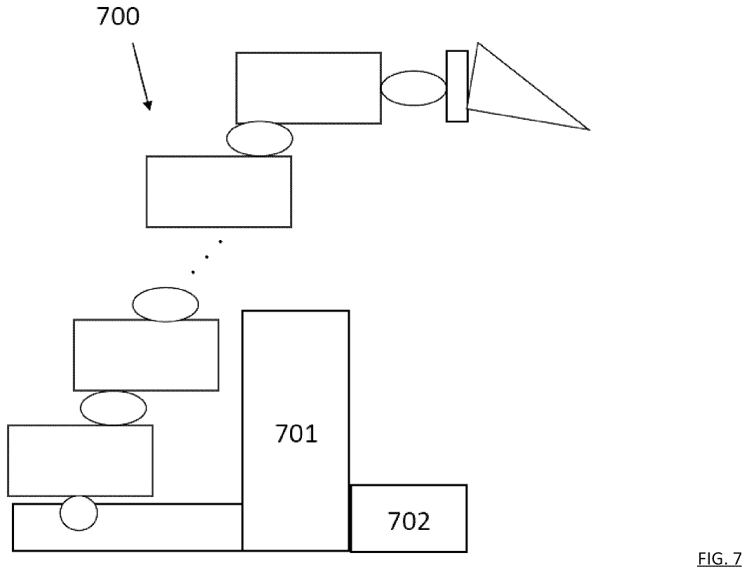

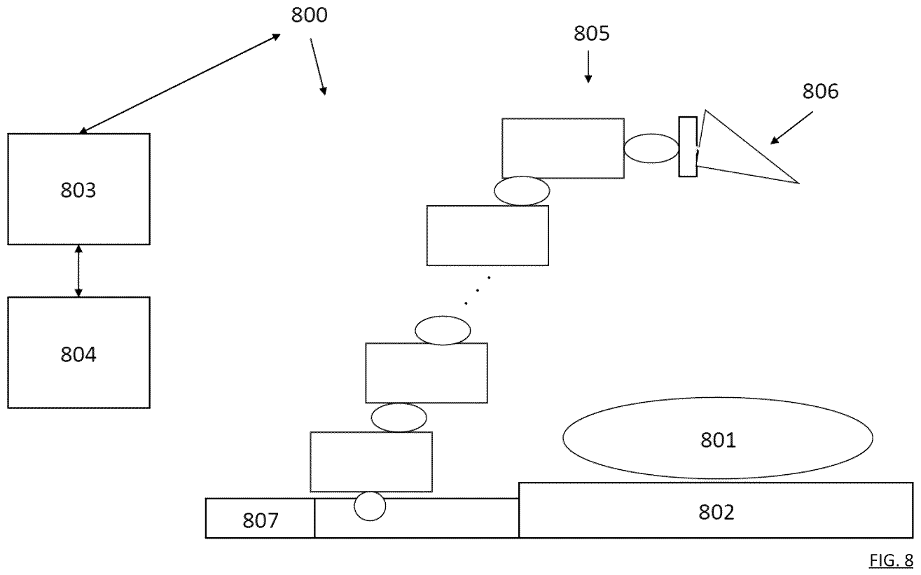

In FIG. 6A, an example robot arm or manipulator is shown raised or tilted/angled back, together with its base attachment, according to an embodiment. In some implementations, a solenoid 601 is located in a portion of the base 602 of the robot 600 or other attachment location, so that when the electric current ceases, the effective magnet behavior of the solenoid 601 ceases, and the portion having the solenoid 601 as an attachment ceases to be attached (e.g., to a base attachment). For example, when the solenoid 601 ceases to act as an attachment method for the base 602 to the underlying support structure, the base 602 can lift up or disengage from another portion of the base 602 and/or a robot base attachment 603, allowing the robot arm or manipulator to move away from the entity 604 in a safe manner. In FIG. 6A, the solenoid 601 acts as an attachment method for a portion of the robot base attachment 603 to a support, table, standalone base, or moveable carriage. The solenoid 601 also can include a screwed hinge, spring attachment, or other attachment which allows the robot arm or manipulator to angle back or tilt or otherwise relocate away from the entity 604. In an embodiment, the robot base 602 tilts away from the support or other base.

FIG. 6B shows a block diagram visualizing a robot arm before tilting/moving/angling away from a base, according to an embodiment. FIG. 6B shows a robot arm 607, a base 608, and a joint 609 (e.g., including a solenoid) when the robot arm 607 has not titled/moved/angled away from the base 608. FIG. 6B may represent a scenario in which the overall assembly (including the robot arm 607, the base 608, and the joint 609) is locked in place (e.g., by the solenoid or other locking/brake mechanism). The configuration of FIG. 6B can also represent a scenario in which a stop condition (e.g., a loss or deliberate removal of power) has not occurred. FIG. 6C shows a block diagram visualizing the robot arm of FIG. 6B after tilting/moving/angling away from the base, according to an embodiment. FIG. 6C shows the robot arm 607 tilted/moved/angled away from the base 608, pivoting at joint 609. FIG. 6C may represent a scenario in which a stop condition (e.g., loss or deliberate removal of power) has occurred, such that one or more components of the assembly can translate and/or rotate.

FIG. 6D depicts a joint region of a robot assembly, in which a robot arm is tilting/moving/angling away from a base, according to an embodiment. Component 610 is part of and/or attached to a base, while component 611 is part of and/or attached to a robot arm. When a stop condition is met, component 612 can move/tilt/rotate such that component 611 and/or the robot arm tilts/moves/angles away from component 610. For contrast, FIGS. 6E and 6F depict the joint region when the robot arm is not titled/moved/angled away from a base, according to an embodiment. As shown in FIG. 6D, component 612 in FIGS. 6E and 6F has not moved/titled/rotated away from component 610.

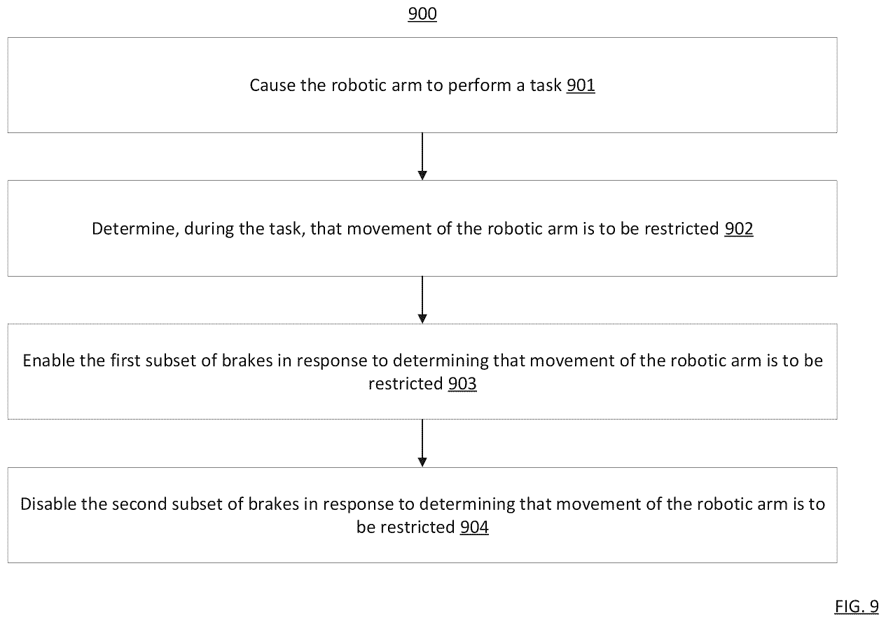

In FIG. 7, a limiting device 701 (e.g., mechanical barricade) can be used to prevent a robot 700 from causing additional undue harm in the event of an error or unexpected loss of power while working on an entity, while undergoing maintenance, undergoing cleaning, and/or the like. In some implementations, the limiting device 701 can restrict the maximum space by stopping or causing to stop all robot motion and is independent of the controller. In some implementations, the limiting device 701 is associated with a support structure 702 (e.g., blockage, wall, floor, gantry, ceiling) near the robot. The support structure 702 can assist with maintaining a position and/or orientation of the limiting device 701. In some implementations, the limiting device 701 can be made of metal, plastic, or any other hard material which is always present at the support structure—whether independent from the robot 700 or attached to the bottom portion of the robot 700. In some implementations, the limiting device 701 can be made of inflatable material, which inflates upon a sensed loss of power, error, or other event, and prevents the robot 700 from moving or falling completely on an entity.

In FIG. 8, an embodiment of a robot device 800 having a robot arm 805 acting upon an entity 801 is shown, where the entity 801 is lying down on a support structure 802 such as a table or other horizontal support structure. Actions of the robot arm 805 are controlled by a controller 803 operatively coupled to a memory 804, the controller 803 providing electronic instructions to the arm 805 of the robot device 800 to make contact, via an attached end effector 806, with the entity 801 and to effect/perform one or more actions. The one or more actions can include, e.g., a massage, a treatment of a specific area of the entity, and/or a resistance testing of a specific area of the entity. The entity 801 can be, for example, a person, an animal, a soft body, or an inanimate object. In this embodiment, an electronic shutdown is caused by the controller 803, the controller 803 receiving a command from a sensor or electronic device 807 measuring and/or providing data to the controller about the robot device 800 or the entity 801. In this embodiment, the entity 801 or another can effect an electronic shutdown of the robot device 800 via a remote control switch which connects to the controller 803 of the robot device 800. The remote control switch can be wired, Bluetooth® enabled, and/or a device software app soft switch enabled over LAN or WLAN network connection.

One or more embodiments provide for one or more disabling features of the robot, which can be used in conjunction with or triggered by an electronic shutdown of the robot. For example, a robot device is disabled by an entity or another manually with a safety mechanical switch or lever associated with at least one of the joints and/or joint brakes connecting and/or connected between the robot arm links. For example, the safety mechanical lever can be a lever piece which can be moved upward or downward, depending upon the type of system. The safety mechanical lever manually releases a brake on the respective joint to which it is attached. The brake on the joint is what locks the respective joint so that the robot is configured to maintain itself in a rigid manner upon error and/or loss of power and does not fall down onto the entity or damage the robot itself by hitting into another device.

In an embodiment, the brake on the respective joint is electromechanical and is controlled by at least one of: the controller commanding a locking or an unlocking of the joint brake and/or a use of a mechanical level or switch to physically disable the joint brake.

In an embodiment, if the robot is powered down either intentionally or unintentionally, at least one or more of the joints of the robot arm are not braked or locked. This can allow a person having the massage or a person nearby to manually move the robot arm away from the body to allow the person to depart safely from the treatment area. In an embodiment, a mechanical or electro-mechanical brake of the wrist of the robot is removed so that it cannot be enabled. In an embodiment, a mechanical or electro-mechanical 5th brake (relative to the base) of a 6-axis robot is removed so that it cannot be enabled in the event of a loss of power. In an embodiment, a mechanical or electro-mechanical 6th brake (relative to the base) of a 7-axis robot is removed so that it cannot be enabled in the event of a loss of power.

In an embodiment, an override of the at least one brake is effected by at least one of a safety-rated force sensor, contact sensor, or soft/hard button.

In the course of the interaction protocol, it may occur that the person becomes uncomfortable. In such case, a switch, button, or sensor can be used by the person or operator or automatically by a sensor acting on stored threshold data to interrupt the interaction protocol being effected.

In some implementations, the software-defined interface (e.g., tablet/device) or stop button is directly attached to the robot device to allow for switch off, and/or engagement or disengagement of brakes by application of or stopping of power via a portable power or other power source, depending upon the brake type employed at specific brake joints of the robot.

Some implementations can use additional safety features such as warning lights, audible alarm or music to alert or identify an error or change in situation. In some implementations, additional safeguarding techniques can be employed, including limiting devices, sensors, fixed barriers, and interlocked barrier guards, flashing lights, signs, whistles, and horns. For example, additional safeguarding techniques can be attached to the robot device and/or communicate with a controller associated with (e.g., included in) the robot device.

In an embodiment, the robot has at least one sensor on its outer surface—directed away from the entity being worked on by the robot—to detect when an operator or another is approaching and/or within the robot's working envelope, to effect a “slow” speed or restriction of movement. In an embodiment, the robot has at least one sensor on its outer surface—directed away from the entity being worked on by the robot—to detect when an operator or another is approaching and/or within the robot's working envelope, to trigger an inflation device surrounding the robot and preventing an operator from entering a critical working area of the robot, e.g., should the power cease unexpectedly or the robot experience a malfunction. In an embodiment, the at least one sensor includes a presence sensing device (e.g., camera, motion detector, etc.).

Embodiments can be used with each other in the same system in order to provide additional redundancy for a safer system.

As described herein, some robot arms/manipulators employ a braking system that can only be disabled electronically. For those robots using power-on electromagnetic brakes, such brakes are only or can only be “on” when the power is on. This means, that when the power is off, the brakes disengage and all joints loosen. This can cause damage to the robot, as well as any persons nearby, in the event of an unexpected power loss. A battery or capacitive storage associated with the robot joints can allow for a slow decrease of power, in the event of power loss from the main source. A cushion, as described herein, can be used to catch or soften the unexpected fall of the robot. For those robots using power-off electromagnetic brakes, such brakes are only or can only be “on” when the power is off. This means, that when the power is off, the brakes lock causing the robot to be immovable. The various embodiments regarding this type of brake is described above in, for example, FIGS. 1 and 2. Some robot arms/manipulators employ a braking system that can be manually released, and employ a manual release lever that allows a person to override the brake when there is no power. In some implementations, a manual release lever is added to at least the brakes located at the wrist joint, the second to last joint, or another joint, to allow for manual movement of the robot arm so that a person can depart safely, but also prevent the heavy robot arm from falling or partially falling on the person. The robot arm joints allow for not only lateral movement, but also vertical movement, which must be taken into account when adding a manual release brake to the robot arm.

Other brakes that can be employed are spring engaged brakes which lock when the power is off, and thus, can be manipulated in systems of at least some implementations as described herein. Permanent magnet brakes in the robot arm joints can allow for usable brakes when the system is on and off, since the magnetic field can continuously flow, and can force parts of the brake to engage in a frictional relationship with the joint, causing the brake to manually lock.

In embodiments, a portable power source could be a battery, such as a 24 volt battery or other size depending upon the robot type and size.

In an embodiment, the table (or work table or surface) under the body or object is able to articulate the different parts of the body independently. Throughout the specification, the surface or structure supporting the body or object is referred to as “table” for ease of reference in embodiments but is not meant to be limited to a table. The supporting structure or surface can be the ground, a chair or other furniture, a highly adjustable table, or other structure or means of support. The support structure can include a sensor, a button or other device which disables the robot device. The support structure can include a mechanical structure which allows an operator or body to physically move the robot away via a track that is unlocked. The support structure can include a mechanical safety block that resides there permanently or inflates or somehow appears during a detected error situation. In embodiments, a system, method, and apparatus provide for a therapeutic massage plan applied by a robot with one or more arms, to the body of a human lying prone, face down, on a massage table. The robot arm(s) can be positioned to the side of the table such that the workspace reach of each robot arm substantially covers the target regions of the therapeutic massage. In an embodiment, the mounting position of the robot arm(s) is determined to increase and/or maximize the pressure application required for the therapeutic massage. In embodiments, the therapeutic massage or other plan can be effected on the front region or a side or other region of a body. In embodiments, the therapeutic massage or other plan can be effected on a body which is human, mammal, animal, or a non-living object. In embodiments, the therapeutic massage or other plan can be effected on non-living soft body objects. Accordingly, it can be desirable in some instances to provide electromechanical safety features for protecting the entity receiving a massage, as described in various embodiments herein.

FIG. 9 shows a flowchart of a method 900, according to an embodiment. In some implementations, method 900 can be performed using an apparatus that includes a base (e.g., base 101, 201, or 602). The apparatus can further include a robotic arm (e.g., arm 102, 202, 402, or 805). The robotic arm can be operatively coupled to the base via a connector (e.g., connector 111 or 211). The robotic arm can include a set of links (e.g., links 103a-n, 203a-n, or 403a-n) interconnected by a set of joints (e.g., joints 104a-n, 204a-n, or 404a-n). A first link (e.g., link 103n, 203n, or 403n) from the set of links can be operatively coupled to the connector. Each joint from the set of joints can include a brake from a set of brakes. Each brake from the set of brakes can be enabled (e.g., locked, braked) or disabled (e.g., unlocked, not braked). The apparatus can further include an end effector (e.g., end effector 105, 205, 407, 806) operatively coupled to the robotic arm. For example, the end effector can be operatively coupled to the robotic arm via a second link (e.g., link 103a, 203a, 403a) from the set of links different from the first link. The end effector can be directly coupled to the second link, or coupled to the end effector via one or more intermediate components (e.g., device 112, sensor 106, device 212, sensor 206, manipulator 405, or wrist 406). The apparatus can further include a controller communicably coupled to at least one of the base, the robotic arm, or the end effector. The controller can be configured to perform method 900. In some implementations, the apparatus can include a support structure that is configured to support an object associated with a task to be performed by the robotic arm. The support structure can be attached to the base. In some implementations, the apparatus can include a mechanical switch (e.g., button, lever, etc.) and/or a software-defined interface (e.g., tablet, phone, remote, etc.) operatively coupled to the base, the robotic arm, the end effector, and/or the controller.

At 901, the robotic arm is caused to perform a task. The task could be, for example, a massage, medical procedure, assembly, and/or the like. Causing the robotic arm to perform the task can include the controller sending an electronic signal representing the task to be performed to one or more components of the robotic arm. In some implementations, the task includes causing the end effector to make contact with and/or come in close contact (e.g., within 1 inch, within 6 inches, within 12 inches, etc.) with a person and/or a deformable body.

At 902, a determination is made, during the task, that movement of the robotic arm is to be restricted. Movement of the robotic arm being restricted can refer to, for example, restricting all or some movements of the robotic arm. Examples of situations that can cause the determination to be made can include, for example, low power, no power, reduction in power to below a predefined power threshold value, a selection by a user via a mechanical button, a selection by a user via an electronic device, a detected malfunction, a sensor reading outside an acceptable range, an undesirable environmental condition, an operator getting too close to the robotic arm (e.g., within 5 feet, within the normal working area of the robotic arm, etc.), and/or the like. In some implementation, the determination that movement of the robotic arm is to be restricted can be made based on a human using the mechanical switch and/or the software-defined interface to request that the movement of the robotic arm be restricted.

At 903, a first subset of brakes is enabled in response to determining that movement of the robotic arm is to be restricted. The first subset of brakes can be included in the set of brakes. Enabling the first subset of brakes can cause each brake in the first subset of brakes to be locked/braked. The first subset of brakes can include, for example, all brakes except the brake furthest from the base (i.e., the closest brake to the end effector) and/or the brake second furthest from the base (i.e., the second closest brake to the end effector). Alternatively, the first subset of brakes can include, for example, all brakes except the brake furthest from the base (i.e., the closest brake to the end effector), the brake second furthest from the base (i.e., the second closest brake to the end effector), and the brake third furthest from the base (i.e., the third closest brake to the end effector), for example to facilitate movement control in three directions (e.g., cartesian x, y, and z directions). In some implementations, 903 can happen automatically (e.g., without requiring human intervention) in response to 902. In some implementations, the first subset of brakes are enabled by receiving a signal sent from the controller.

At 904, a second subset of brakes are disabled in response to determining that movement of the robotic arm is to be restricted. The second subset of brakes are included in the set of brakes. The second subset of brakes are different than he first subset of brakes. Disabling the second subset of brakes can cause each brake in the second subset of brakes to be unlocked/not braked. The second subset of brakes can include, for example, the brake furthest from the base (i.e., closest brake to the end effector) and/or the brake second further from the base (i.e., second closest brake to the end effector). The second subset of brakes can include all brakes from the set of brakes that are not included in the first subset of brakes. In some implementations, the second subset of brakes are disabled by receiving a signal sent from the controller. In some implementations, 904 can happen automatically (e.g., without requiring human intervention) in response to 903. In some implementations, 904 and 903 can occur at substantially the same time (e.g., within 0.1 second of, within 0.5 second of, within 1 second of, within 2 second of, etc.).

In some implementations of method 900, the task includes causing the end effector to make contact with a person and/or a deformable body, and disabling the second sub of brakes facilitates movement of the end effector away from the person and/or the deformable body (e.g., by the person, the deformable body, by a different person, etc.).

In some implementations of method 900, the task includes causing the end effector to make contact with a person and/or a deformable body, and enabling the first subset of brakes and disabling the second subset of brakes: (1) minimizes an amount of unfixed rotational inertia associated with the robotic arm, (2) minimizes (or at least decreases) an amount of unsupported weight of the robotic arm on the person and/or the deformable body, and/or (3) minimizes (or at least decreases) an amount of force sufficient to break contact between the end effector and the person and/or the deformable body.

The apparatus performing method 900 is not limited to having one robotic arm (see, e.g., FIG. 4). The apparatus can include, for example, two or more robotics arms. Therefore, in some implementations of method 900, the base is a first base, the robotic arm is a first robotic arm, the connector is a first connector, the set of links is a first et of links, the set of joints is a first set of joints, the set of brakes is a first set of brakes, the end effector is a first end effector, and the task is a further task. The apparatus further includes a second base, which can be separate from or connected to the first base. The apparatus further includes a second robotic arm operatively coupled to the second base via a second connector. The second robotic arm can be separate from the first robotic arm. The second robotic arm can include a second set of links interconnected by a second set of joints. A first link from the second set of links can be operatively coupled to the second connector. Each joint from the second set of joints can include a brake from a second set of brakes. The second set of brakes can include a third subset of brakes and a fourth subset of brakes. Each brake from the second set of brakes can be configured to be enabled or disabled.

In an embodiment, an apparatus comprises a base (e.g., base 101, 201, or 602). The apparatus further comprises a robotic arm (e.g., arm 102, 202, 402, or 805) operatively coupled to the base via a connector (e.g., connector 111 or 211). The robotic arm includes a set of links (e.g., links 103a-n, 203a-n, or 403a-n) interconnected by a set of joints (e.g., joints 104a-n, 204a-n, 404a-n). A first link (e.g., link 103n, 203n, or 403n) from the set of links is operatively coupled to the connector. Each joint from the set of joints includes a brake from a set of brakes, each brake from the set of brakes configured to be enabled (e.g., locked, braked) or disabled (e.g., unlocked, not braked). The apparatus further comprises an end effector (e.g., end effector 105, 205, 407, or 806) operatively coupled to the robotic arm via a second link (e.g., link 103n, 203n, or 403n) from the set of links different from the first link. The end effector can be directly coupled to the second link, or coupled to the end effector via one or more intermediate components (e.g., device 112, sensor 106, device 212, sensor 206, manipulator 405, or wrist 406). The apparatus further comprises a controller, communicably coupled to at least one of the base, the robotic arm, or the end effector. The controller is configured to cause the robotic arm to perform a task, and determine, during the task, that movement of the robotic arm is to be restricted.

In some implementations, at least one brake from the set of brakes is freely removable by a human (e.g., human the task is being performed on, an operator, or any other human) such than an orientation or a position of the end effector is modifiable (e.g., human the task is being performed on, an operator, or any other human). For example, the human can remove the at least one brake and push and/or pull the robotic arm to modify the orientation and/or position of the robotic arm and/or the end effector.

In some implementations, the set of brakes include at least one friction brake, and an orientation and/or position of the robotic arm, when the set of brakes are enabled and/or disabled, does not change due to gravity (e.g., stays in place without other external forces), and (2) does change due to force being provided by a human to the robotic arm.

In some implementations, the at least one brake from the set of brakes is configured to allow at least one joint form the set of joints to move in a first direction but not a second direction different than the first direction. For example, the second direction could be all other directions different than the first direction. The first direction can be linear and/or rotational.

In some implementations, the controller is further configured to send a signal to cause at least one brake from the set of brakes to be configured in one of a first mode or a second mode. In the first mode, the at least one brake is configured to allow at least one joint from the set of joints to move in a first direction but not a second direction different than the first direction. In the second mode, the at least one brake is configured to allow the at least one joint to move in the second direction but not the first direction. The first direction can be linear and/or rotational. The second direction can be linear or rotational.

In some implementations, the controller is further configured to cause generation of an alarm in response to determining that movement of the robotic arm is to be restricted. For example, the alarm could be audible and/or visual. The alarm can be generated at the base, the robotic arm, the end effector, a remote compute device, a local compute device, and/or the like.

Any number of joints can be included in the plurality of joints (e.g., 2, 3, 4, 5, 6, 7, 8, 9, 10, etc.). In some implementations, the plurality of joints includes at least six joints. In some implementations, the plurality of joints includes at least seven joints.

In some implementations, determining that movement of the robotic arm is to be restricted is based on a reduction in power (e.g., at the apparatus and/or the robotic arm) to below a predefined power threshold value. The apparatus can be configured to automatically reposition the base (e.g., without requiring human intervention) in response to the power being below the predefined power threshold value.

In some implementations, the task causes the end effector to make contact with a person and/or a deformable body, and at least one brake from the set of brakes, when disabled (e.g., mechanically and/or electronically), facilitates movement of the robotic arm away from the person and/or the deformable body. For example, the at least one brake, when disabled, allows the person or a different person nearby modify a position and/or orientation of the robotic arm away from the person and/or deformable body.

In an embodiment, a non-transitory, processor-readable medium storing code representing instructions executable by a processor. The code comprises code to cause the processor to cause a robot to perform a task that includes causing an end effector (e.g., end effector 105, 205, 407, or 806) included in the robot to contact an object. The robot includes a set of links (e.g., links 103a-n, 203a-n, or 403a-n) interconnected by a plurality of joints (joints 104a-n, 204a-n, or 404a-n). Each joint from the plurality of joints includes a brake from a plurality of brakes. Each brake from the plurality of brakes is configured to be enabled (e.g., locked, braked) or disabled (e.g., unlocked, not braked). The plurality of brakes include a first set of brakes and a second set of brakes. The end effector is coupled (e.g., directly or via one or more intervening device) to at least one of a link from the set of links or an attachment device coupled to the link from the set of links. The code further comprises code to cause the processor to determine, during the task, that movement of the robot is to be restricted. The code further comprises code to cause the processor to enable the first set of brakes in response to determining that movement of the robot is to be restricted.

The code further comprises code to cause the processor to disable the second set of brakes in response to determining that movement of the robot is to be restricted.

In some implementations, the code to cause the processor to determine that movement of the robot is to be restricted includes code to cause the processor to determine that movement of the robot is to be restricted based on received sensor data associated with a safety condition. The sensor data could be collected by, for example, a camera, a pressure sensor, a distance sensor, a motion sensor, a power sensor, and/or the like. The safety condition could be, for example, power less than a threshold, distance less than a threshold, pressure greater than a threshold, and/or the like.

In some implementations, the object is a human and the code to cause the processor to determine that movement of the robot is to be restricted includes code to cause the processor to determine that movement of the robot is to be response in response to a selection from the human (e.g., via a switch, a button, a lever, a selection on a compute device, and/or the like). Features of the various embodiments of the above-identified system and method described herein can be modeled and/or effected and/or controlled by a general computer, special purpose computer, a processor, and a smart device having a processor. Embodiments of the method instructions can be stored on a computer-readable medium, the medium being virtual or hardware or portable or in the cloud/networked, having instructions thereon which are readable or can be made to be readable by a computer or processor so that the computer software instructions can be executed. The various embodiments described herein, and those equivalents thereto, can be used for a variety of nonanalogous objects, e.g., human body, animal body, soft body having deformable characteristics, a nonhomogenous body having soft and hard features. The various embodiments described herein, and those equivalents thereto, can be used for massage applications, sensing applications, modeling applications, and others.

### Robotic Manipulator Dampening for Electromechanical Safety

The following are embodiments of robotic manipulator dampening for electromechanical safety. In some embodiments, for safety, the robotic arms are configured to automatically fall or move away or otherwise withdraw away from the massage subject or massage table or robotic massage platform in response to an e-stop being triggered or some other (e.g., emergency) indication by the user to release the robotic arms. For example, in some industrial applications, when a stop event occurs for a robot, the robot's motion is frozen, such that the robot is in a locked state. While such freezing or locking of a robotic manipulator may be beneficial in contexts such as industrial applications, in the context of a robotic massage, in which a robotic arm is being used to apply force to a subject such as a person, complete freezing of all motion of the robot may be an undesirable behavior. For example, in a robotic massage context, when an emergency stop event occurs (also referred to herein as an e-stop event), all forces on the subject should be removed. If an e-stop event occurs while the robotic arm is in the middle of applying force to the subject, completely freezing the motion of the robotic arm could make it challenging for the robot to be moved away from the massage subject, as, for example, the robotic arm itself has weight. Using embodiments of the safety techniques described herein, to prevent such a situation, mechanisms are provided such that in the event of an e-stop, the robotic arms are configured to automatically move away from the massage subject (and remove forces on the subject due to the robotic arms).

The following are embodiments of controlling such robotic arm withdrawal behavior. As will be described in further detail below, dampening is performed to control such robotic arm withdrawal behavior.

In some embodiments, the robotic massage system includes a robotic base assembly that includes a base joint that determines how a base plate (to which the robotic arm is attached) moves away from the robotic massage table. In some embodiments, the base joint is positioned next to the platform. In some embodiments, the base joint is configured such that it is limited to allowing the base plate (and thus the robotic arm attached to the base) to move away from the robotic massage platform. In some embodiments, the base joint is a rotating joint that, when opened, allows the robotic arm to tilt away from the robotic massage platform. In some embodiments, the robotic massage system includes a locking mechanism, such as an electromagnetic locking mechanism, to lock the base joint in position (e.g., to prevent the base joint from opening during normal operation).

In some embodiments, the robotic massage system includes a spring and a damper. In some embodiments, in an e-stop event, the spring actively lifts the robotic arm away from the massage table (e.g., by causing the base joint to open, which then tilts the base plate, including the robotic arm attached to the base plate). In some embodiments, the damper is used to regulate the speed at which the arm is moved away from the massage table. Further details regarding controlled withdrawal behavior of robotic arms for safety in a robotic massage system are described below.

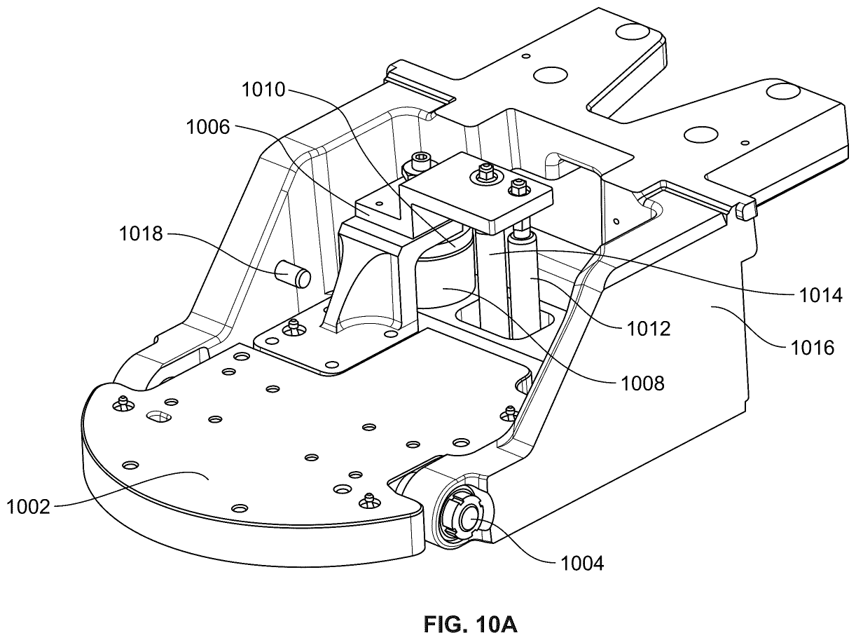

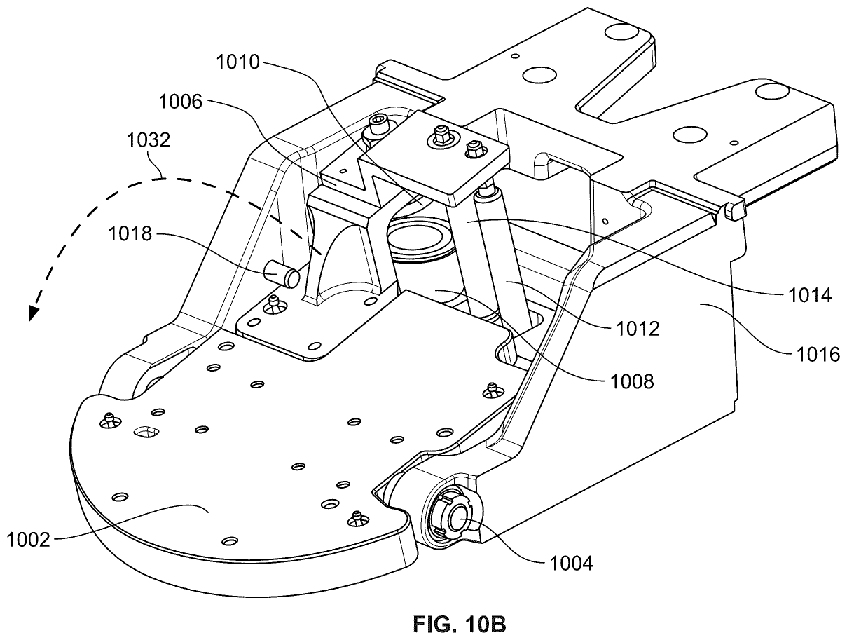

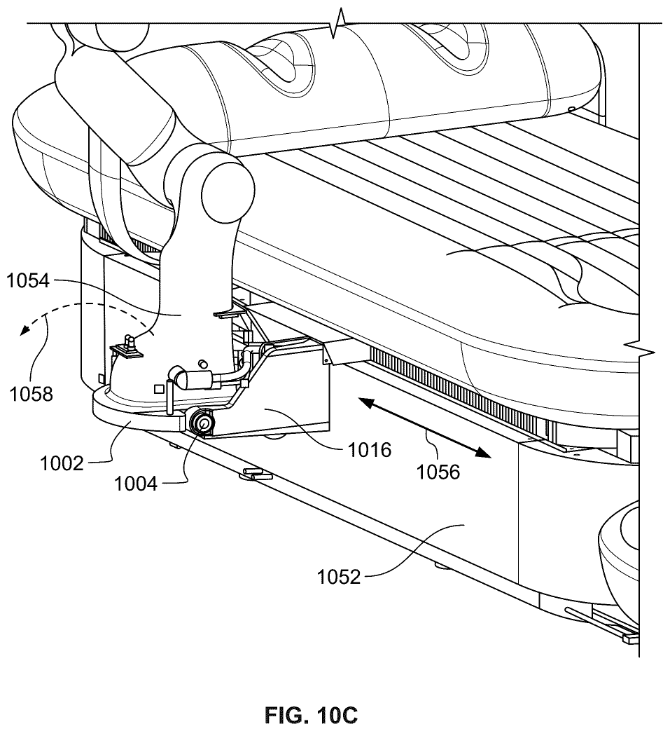

FIG. 10A illustrates an embodiment of a robot base assembly. As one example, the robot base assembly 1000 is an embodiment of base 608 of FIG. 6C. In this example, a state of the robot base assembly during regular operation is shown. In this example, portion 1016 of the robot base assembly connects the base assembly to a robotic massage table of a robotic massage system. In this example, a robotic arm (e.g., robotic arm 607) sits on or is otherwise coupled or attached to arm base plate 1002. In this example, the base plate is able to tilt about rotating base joint 1004, which includes a shaft. In this example, the base joint 1004 determines how base plate 1002 moves relative to portion 1016, which connects the robotic massage platform (and thus the base joint determines how the robotic arm moves relative to the massage table). As shown in this example, the arm base plate is also coupled to a locking bracket (1006) (also referred to herein as a magnet bracket, or magnetic catch). For example, the arm base plate and the locking bracket move together. In this example, electromagnet (1008) (such as a solenoid) is a component used to lock or hold the locking or magnet bracket in place. For example, the magnetic bracket 1006 includes a receiver plate 1010 for the electromagnet 1008. When engaged (e.g., with power), the electromagnet 1008 is attracted to portion 1010 (magnet receiver plate) of the locking magnetic bracket, thereby locking the arm base plate in place by preventing the arm base from being able to rotate about the rotating joint. As shown in this example, the electromagnetic lock, when engaged (e.g., with power) and in the lock state, rigidly locks the locking bracket (and thus the arm base plate) in place during regular operation (to prevent the arm from being moved away from the massage table during the normal course of operation by preventing the arm base plate from rotating about the base joint 1004). The use of an electromagnet for locking provides various benefits. For example, if the electromagnet fails, it will fail in the open condition, releasing the magnet bracket 1006 from the portion 1016 of the base assembly connected to the table, allowing the arm to fall away. This is in contrast to other locking mechanisms that fail in a locked position, which could be detrimental in the context of a robotic massage system.

As shown in this example, the base joint is a rotating joint that allows the base plate (and thus the arm) to tilt away from the massage table. Under regular operation (as shown in the example of FIG. 10A), the base joint is in a closed state. The base joint is locked in the closed state when the electromagnetic lock is engaged, preventing the base joint from opening. When an e-stop event occurs, the electromagnetic lock is disengaged or released. When disengaged, the base joint is allowed to open. In some embodiments, the base joint is configured or otherwise limited to rotating in one direction (e.g., in the direction in which the arm base is rotated away from the massage table). For example, while the joints of the robotic arm are allowed to move in various directions, the base joint is only allowed to open in a manner that causes the base plate to tilt or otherwise move away from the massage subject or massage table/platform that supports the massage subject. Providing such a limit on the directionality of opening of the base joint ensures that when the power is cut (e.g., electrical power to the system is commanded to go off during an e-stop event), the robot base assembly has a behavior in which the arm will only fall or move away from the person (and not in the direction of the subject). For example, the robot base assembly is biased to cause opening of the base joint in a manner that causes the robot arm to move away from the massage subject.

In this example, the robot base assembly includes spring 1012. In this example, the spring is an active component that is actively attempting to push the robotic arm away from the massage subject (e.g., by pushing against the locking bracket 1006). For example, under regular operation, the spring is in a compressed state, where the locking of the base by the electromagnetic lock counters the spring action. In this example, during regular operation, the spring is pushing with an upwards force. The electromagnet, when engaged/activated, counteracts and overcomes the force of the spring to hold the locking bracket and arm base plate in place (and also keeps the base joint closed, or otherwise prevented from opening).

When the electromagnetic lock is disengaged during an e-stop event (e.g., by removing power from the electromagnet), the spring (which offsets the weight of the robotic arm) is allowed to uncompress, which pushes the locking bracket 1006 and causes the base joint to open, resulting in the base plate 1002 (with the attached arm) tilting away from the massage subject. This tilting away of the base plate about the shaft of the rotating base joint allows the robotic arm to fall away from the massage subject, relieving any forces or pressures on a user's body that had been being applied by the robotic arm, and allowing the massage subject to leave the massage table.

In this example, the system includes damper 1014. The damper regulates, controls, or otherwise slows down the speed at which the robotic arms fall away. The use of the damper to slow down the movement of the arm away from the massage subject improves safety of the robotic massage system. For example, if the damper were not present, and there were only the spring, release of the spring may result in a snappy or fast motion due to reversal of the forces that had been applied to now being in the opposite direction. If, for example, the arm is caught on a subject's clothing or necklace, the sudden movement away of the arm can result in a safety hazard. Sudden motion of the arm away from the massage table can also pose a safety hazard to the surroundings of the massage table. For example, if another person is in the room standing near the robotic arm, then the sudden motion of the robotic arm away from the table in an e-stop event may cause the robotic arm to hit the person in the surrounding area as the arm moves away from the table. As another example, without the speed reduction mechanism such as the damper described herein, if the robotic arm were to fall away suddenly, when it hits an end stop assembly (e.g., end stop pin 1018), this can result in a potentially severe impact to the robotic arm that could also damage equipment.

In this example, the damper component is a speed-limiting damper. In the example shown, the damper sits in parallel with the spring. In this example, the damper provides a counter or braking force to the force applied by the spring in pushing the locking bracket (and causing the bracket/base plate to rotate about the rotating joint). As one example, as speed increases, the damper provides an increased braking force. The use of the damper, as configured herein in the example robot base assembly shown in FIG. 10A, allows the withdrawal motion of the robotic arm (tilting away in this example involving a rotating base joint) to be a smooth, slower motion, rather than a sudden motion, thereby further enhancing safety and removing potential safety hazards in an e-stop event. Further, performing such dampening allows the robotic arm to gracefully and smoothly fall away, preventing a sudden impact when the robotic arm reaches an end stop, thereby also preventing damage to the robotic arm. Thus, embodiments of the dampening described herein provide various benefits, such as providing a safe robotic massage experience by protecting the massage subject and people in the surrounding vicinity of the robotic massage system, as well as protecting equipment from damage.

FIG. 10B illustrates an embodiment of a robot base assembly. In the example of FIG. 10B, the robot base assembly of FIG. 10A is shown in an open or moved-away state, such as during an e-stop event. For example, the robot base assembly is in the state shown in FIG. 10B responsive to an e-stop event. As shown in this example, the robot arm base plate 1002 is rotated about the shaft of the base joint 1004. For example, suppose an e-stop event is activated (e.g., by the user). In response to the e-stop event, power is removed from the electromagnet 1008. This causes electromagnet 1008 to no longer attract magnet receiver plate 1010. Now there is no longer the electromagnetic force countering the force of spring 1012. The spring 1012 then pushes the magnet bracket 1006 (where there is now a gap between the magnetic receiver plate 1010 and electromagnet 1008 shown in the example of FIG. 10B), causing the arm base plate (and thus the robotic arm attached to the base plate) to tilt about the shaft of rotating base joint 1004 in the direction indicated by 1032. In some embodiments, when rotating, the rotating joint opens from a starting operating position such as the state of the robot base assembly shown in FIG. 10A (e.g., where the receiver plate 1010 and electromagnet 1008 are in contact and locked), with an ending position determined, for example, by an end stop. One example of an end stop is a physical end stop such as end stop pin 1018. In other embodiments, the spring and/or damper contain a stop that restricts how much the pivoting joint can open/rotate.

FIG. 10C illustrates an embodiment of a robot base assembly in a robotic massage system. In this example, a robot base assembly such as that shown in FIGS. 10A and 10B is shown attached to a robotic massage table/platform 1052. In this example, portion 1016 of the robot base assembly is coupled to massage platform 1052, and travels along the table (directions of travel indicated at 1056), while holding the robotic arm 1054. As described above, the robotic arm 1054 sits on, or is otherwise coupled to base plate 1002. As shown in this example, the base plate (along with the attached arm) rotates or tilts around the shaft of rotating joint 1004. As shown in this example, during an e-stop event, using embodiments of the techniques described herein, the robotic arm is caused to tilt or fall away (about the shaft of the base joint) from the massage table in the direction indicated at 1058. In some embodiments, dampener 1008 limits or otherwise controls or regulates the speed at which the base plate tilts about the rotating joint 1004 (e.g., by providing a braking force countering the force of the spring 1012 that is pushing the bracket 1006 and the base plate 1002).

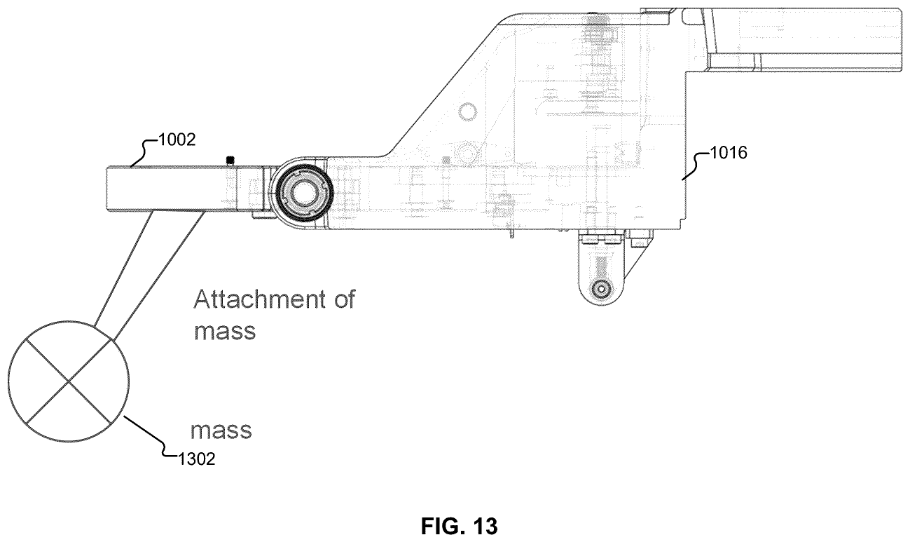

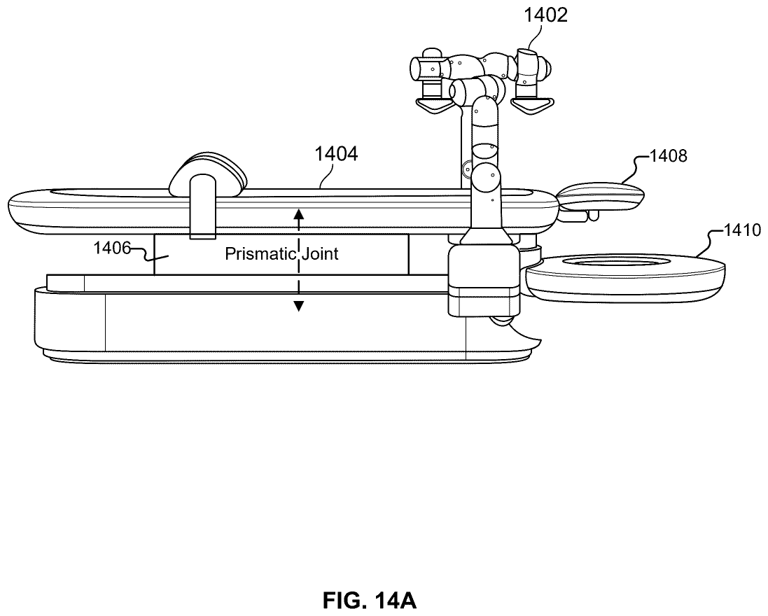

In the above examples, a spring is used to push the robot arm away. For example, given the configuration and placement of the robotic arm, how it is being manipulated during the massage (e.g., stretched out over an entity to perform the massage), its orientation, etc., the robotic arm will have certain characteristics as to how the weight of the robot is distributed (and, for example, hanging over the user). In some embodiments, the spring is used to offset the weight of the arm to push it away. In other embodiments, the robotic arm is configured (e.g., via a different placement of the joints, distribution of weight/mass, different orientation, etc.) such that gravity would perform the work done by the spring (i.e., so that a spring is not included or needed), where the robotic arm is caused to move away from the table due to gravity (with the locking mechanism holding the arm in place during regular operation of the robotic massage to counter the force of gravity). For example, the relative placement of joints to the robotic massage system can be changed to achieve the same or similar functionality by balancing the mass distribution differently (such as with the example shown in FIG. 13). In such an example implementation, the spring may not be needed. For example, the spring force is replaced by having a distribution of mass in the system such that gravity would perform the work of the spring. In this example, where the robotic arm is designed to move away naturally due to gravity (e.g., where gravity would cause the arm to tilt away), a dampener such as dampener 1014 can still be utilized to control the speed at which the arm moves away (e.g., slow down the speed at which the arm moves away or withdraws from the table). In other embodiments, the speed at which the arm moves away can also be controlled by tuning of the distribution of mass of the arm, and the dampener may also not be needed.

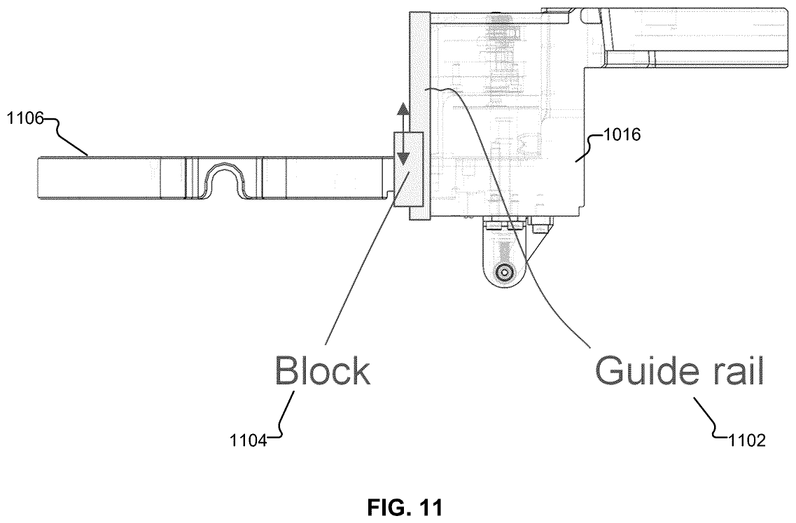

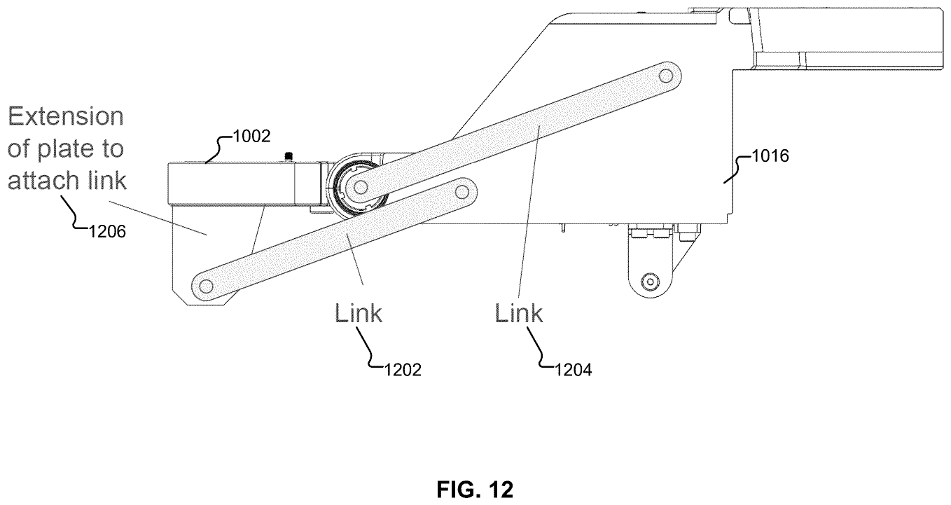

In the above examples of FIGS. 10A-10C, a rotating base joint with a pivoting shaft was shown. The pivoting shaft allows the arm to tilt or rotate away from the massage subject. The techniques described herein may be various adapted to accommodate other types of base joints or openable/closable/adjustable components. Another example base joint is a translating joint that facilitates sliding motions (such as with the example shown in FIG. 11). For example, rather than tilting away, the robotic arm(s) can be caused to have a withdrawal behavior in which the arms are pushed or translated away from the table/user. For example, a spring can be utilized that in normal operation is applying a pushing force to slide the arms away, where the locking mechanism provides a counter force to the spring. When an e-stop event occurs, the locking mechanism is disengaged, allowing the spring to push the arm away from the massage table. The dampener can still be utilized to slow or smooth the moving away of the robotic arms. In other embodiments, joints such as linkages are used that perform both translating and rotating (such as with the example shown in FIG. 12).

The following are further embodiments regarding safety mechanisms that facilitate separation between a robotic arm (e.g., end effector of the robotic arm) and an object that the robotic arm interacts with in operation. In some embodiments, facilitating separation between the robotic arm and the object includes causing one or both of the robotic arm and the object to move away from each other. In some embodiments, the separation mechanism is activated in response to an emergency stop event (e.g., user activation of e-stop). Separation between a robotic arm and an object may be triggered due to other events as well, such as a power outage. The safety mechanisms described herein may be variously adapted to accommodate robotic systems with multiple robotic arms that interact with one or more objects. While examples in the context of robotic massage (with robotic arms interacting with a deformable object such as a human body) are described herein for illustrative purposes, embodiments of the robotic arm and object separation techniques described herein may be variously adapted to accommodate other applications or contexts in which it is desirable (e.g., for safety) to separate a robot from an object that the robot interacts with during operation.

### Embodiments of Component Specification

In the above examples of FIGS. 10A-10C, components 1002, 1006, and 1010 are mechanically mounted together, and move together as rigid bodies. In the example of FIG. 10A, the joint (with shaft) 1004 is shown in its operating position (e.g., closed, and locked in place by the locking mechanism during operation). In FIG. 10B, the base joint is shown in an intermediate open position (where the base, which is also coupled to the rotating/pivoting joint is also rotated from its operating position as a result, when the locking mechanism is released).

The following are further embodiments regarding specification, configuration, tuning, or selection of values for components that facilitate separation between (an end effector of) a robotic arm and an object that the robotic arm (via the end effector) interacts with, such as damping rate of the damper, holding strength or force of the (electro)magnetic locking mechanism, spring rate of the spring, force of the spring when in its operating position, etc. In various embodiments, the specification of component values is based on a variety of factors. For example, the sizing of components may be determined relative to each other, based on safety parameters, timing parameters, etc. Further examples of considerations and factors in sizing components for facilitating separation or displacement (e.g., distance-wise) between a subject and a massage end effector (of a robotic arm) are described below.

### Locking Mechanism Holding Force Sizing

The following are embodiments of specification of holding force of a locking mechanism that is configured to lock the openable component (e.g., pivoting joint) in its operating position.

An electromagnet is one example of a locking mechanism for locking a multi-position component (e.g., that is openable/closable, such as the base pivoting joint described herein). One property of the electromagnet that is beneficial for safety is that an electromagnet is prone to never fail in a locked position. In the configurations described herein, when the electromagnet fails, the magnetic holding force that is locking the base joint is disengaged, and the base joint is allowed to open (and the arm will not be stuck). For example, even when a power outage occurs, or power is cut to the electromagnet, the electromagnet will release its hold on the base joint, allowing the joint to open. This is an improvement over other mechanisms, which may fail in a locked position, causing a user to become trapped. Further benefits of the electromagnet include cost-efficiency and reliability.

In the following examples, the locking mechanism is implemented using a magnet or electromagnet, and the holding strength or force being specified is a holding force of a magnet. In various embodiments, the electromagnetic force specified for the electromagnetic locking mechanism is related or based on the force of the spring when at its operating position, spring rate of the spring, as well as the damping rate of the damper.

During operation, in which a massage is applied to the user (and where the end effector is interacting, and in contact with, the subject), there is a force that is from the user back into the tool/end effector of the robotic arm, that is actively pushing the robotic arm away from the electromagnetic locking mechanism (and is trying to break the electromagnetic connection). The electromagnetic holding force keeps the robotic arm from pivoting away from the user during operation (by locking the pivoting joint in its operating position, which is closed), such as when the robotic arm is manipulating the end effector to be in contact with the user. For example, depending on where over the subject's body force is being applied by the robotic arm, different amounts of force will be projected back to the electromagnetic locking mechanism (which in some embodiments is located at the pivoting joint).

If the opposing force from the user back to the robotic arm (projected back to the electromagnet/pivoting joint at the base of the robotic arm) exceeds the magnetic holding force, then the electromagnetic locking connection will break, and the arm will be allowed to pivot about the base joint (as the joint is no longer locked in its operating, closed position). For example, if during regular operation in which the massage is being applied to the user, the force projected back to the locking mechanism exceeds the lock's holding force, then the lock's hold will break, and the openable component (e.g., pivoting joint) will be able to open). This would disrupt the massage during regular operation.

In some embodiments, to prevent the electromagnetic connection from breaking during operation, the magnetic holding force of the electromagnet is specified based on the amount of force that is applied by the robotic arm for various massage content. For example, the electromagnetic locking mechanism is specified with a higher magnetic holding force to accommodate higher force massage content. For example, the electromagnetic locking mechanism is sized such that the breakaway/holding force (where the electromagnet disengages) exceeds any forces that are projected back to the electromagnetic locking mechanism/pivoting base joint due to the application of massage content.

In some embodiments, for user safety, the holding or breakaway force of the electromagnet is specified or set at a threshold level such that the electromagnet disengages (e.g., passively) when a force applied by the robotic arm exceeds a certain value. As one example, suppose that it is desired for the robotic arm to be able to push down with no more than 200 Newtons of force. In this case, it is not desirable for the electromagnet to fail before (or less than) 200 Newtons of force is applied by the robotic arm, but it would be desirable for the electromagnet to fail or disengage if the 200 Newtons of force is exceeded (or, as another example, no more than 200-400 Newtons or equivalent in pounds, as appropriate). Specifying the holding force in such a manner provides an additional protection or safety mechanism, such that more than a maximum or threshold level of force cannot be applied by the robotic arm to the user. For example, the magnetic holding force is sized such that the magnetic connection will disengage or break when the maximum permitted robotic arm force is exceeded, allowing the robotic arm to be displaced (e.g., pivoted) away from the subject. In some embodiments, the breakaway or maximum holding force (force at which the electromagnet is designed to disengage) is based on characterization of forces applied during the massage and the forces that are translated to the location of the electromagnetic locking mechanism.

In some embodiments, the sizing of the breakaway or disengagement force or maximum holding force for the electromagnet provides a type of passive safety mechanism. For example, suppose that the electromagnet is designed or specified to hold up to 250 Newtons of force. This upper bound is selected to provide locking during expected operation. If this maximum level of force is exceeded, then the maximum holding force of the electromagnet is broken, and the electromagnet disengages. For example, the electromagnet force is tuned such that if the force exerted by the robotic arm on the subject (in applying some massage content) were to exceed a threshold amount of force, then the electromagnetic lock is broken. In this way, excessive amounts of force cannot be inadvertently applied to the user, as the locking mechanism will passively disengage in such scenarios.

In some embodiments, a specified holding force or breakaway force of the magnet will differ from the force of the robot arm above, due to the different mechanical advantage between the robot end effector location and the locking magnet location in the robot base assembly. Examples of magnet holding strength, provided for illustrative purposes and without limitation, are 1250N (Newtons) or 2300N. Other magnet holding force values may be utilized, as appropriate.

In other embodiments, active release mechanisms for electromagnetics are utilized. For example, a pressure sensitive control mechanism is utilized in which if more than a threshold amount of force is detected or sensed, then the electromagnetic locking mechanism is actively released (e.g., power is cut to the electromagnetic locking mechanism, causing the magnet to disengage).

As described above, the applied massage content force produces or results in a force exerted on the electromagnet that the magnet is holding against. Different forces on the magnet will be produced or result depending on where (over the subject body) and what direction the massage force is being applied (as the robotic arm may be in any of a variety of joint configurations in order to have the end effector reach and come into contact with some particular region of the subject). In some embodiments, the maximum holding force of the magnet is sized based on a worst-case scenario, such as a maximum force that would be translated back to the electromagnetic locking mechanism during operation (during which the maximum force is being applied to perform massage content). Such a worst-case scenario can be non-trivial to define, and may depend on the specific configuration of the robot arm as well as the remainder of the system. In some embodiments, the worst-case scenario is with the robot arm at maximum reach and the force from the robot arm directed tangential to the motion of the pivoting joint, while in other situations, a more complex definition of the worst-case scenario may be the case.

In some embodiments, with respect to safety considerations, in the event that the user activates the emergency stop (also referred to herein as “e-stop”), or if there is another emergency condition that occurs (e.g., power outage of the robotic massage system), even if the robotic arms enter into a locked mode (where the joints of the arm are locked in the configuration they were in when the power outage occurred), then the safety mechanisms described herein move the arms away from the user so that the user can leave the robotic massage system (e.g., via removal or relieving of forces on the subject due to the arm).

Further, the user may also physically move the robotic arms away (as the locking mechanism is released). In some embodiments, the robotic massage system is associated with a requirement or parameter comprising a value of the maximum force that is allowed to be required from, or asked of, the user to push on the robotic arm to move it away.

In some embodiments, the maximum force that should be required of the user to move the robotic arm(s) away is determined based on the possible positions that the robotic arm can be in when an emergency stop is triggered. For example, if the emergency stop is triggered when the robotic arm is fully stretched out, due to the force-balance (e.g., where the force from the spring, together with a force provided by the user will need to overcome the mass of the robot arm, such that the total effective moment around the pivoting joint causes the arm to move away from the user) between the spring and the robotic arm in its outstretched position, then it will be slower for the base pivot joint to be opened by the spring (as it is the spring that is pushing against the mass of the arm to open the pivoting joint from its closed, operating position, and move the arm away). On the other hand, if the arm is in a more side-position (e.g., closer to the side of the user or closer to the base), this is a different force-balance, and less force is required from the spring to push the robotic arm away. For example, between the robotic arm being in the side-position versus the outstretched position, it would be more difficult for the spring force to open the arm when the arm is in the outstretched position (and more force would be required from the user to move the arm away in comparison). In some embodiments, the maximum permitted force that can be required or expected of the user is specified as a safety parameter. In some embodiments, the minimum spring force specified for the spring (at its operating position) is then determined as a function of the force needed to move the arm away (e.g., when the arm is fully outstretched), and the maximum force permitted to be required from the user to apply to the end effector. For illustrative purposes, 5 pounds of force is one example value for the requirement for the maximum force the user may need to push with.

In the above embodiments, the robotic massage system is designed to automatically move the arms away from the subject in the event of an e-stop scenario. As described above, there may be some edge cases, such as when the robotic arm is fully reaching out (e.g., full extension of the arm over the subject), in which the user may need to provide some force (e.g., on the end effector) to move the arm/end effector away. The components of the robotic massage system are specified such that any force needed by the user to move the arm away is minimal so that the user can easily leave the massage table. In this way, the user is not required to push with any excessive force (either with their hand, or through their back if the user is in a prone position) to leave the robotic massage table.

### Spring Force/Rate Sizing

In some embodiments, the spring force of the spring (that is pushing to open the pivoting joint at the base to which a robotic arm is attached), such as at its operating position, is determined based on various factors including, without limitation: the requirement for the maximum force that can be asked from the user to push on the arm, the weight of the robotic arm, how the mass of the arm is distributed in different positions/poses, etc.

In the above examples, the spring force is positioned next to the electromagnetic locking mechanism, where the spring is counteracting the electromagnet. For example, the spring force is acting in a direction to open the pivoting joint, while the magnet holding force is acting to hold or lock the pivoting joint in its operating, closed position. In some embodiments, the holding force or strength of the electromagnet is specified to overcome both the force that the spring is producing to lift the arm away, as well as the worst-case force from the massage content.

In some embodiments, to lower the amount of force required from the user to push away the robotic arm in an e-stop event (an example of a safety parameter), the spring force is increased (to increase the lift force provided by the spring to open the pivoting joint, such as in an e-stop event). With the increase in spring force, the electromagnetic locking or holding force is increased as well to counteract and overcome both the spring force and massage content force (projected back to the pivoting joint where the locking mechanism is exerting its holding force) during normal or regular operation. In this example, the spring force is determined based on the desired amount of lift force from the spring to facilitate robot-object separation during an e-stop event, and the maximum force that can be required from the user during the e-stop event. In some embodiments, the magnetic holding force is specified to compensate both for the spring force and the massage content force during operation. In some embodiments, the magnetic holding force specified for the electromagnet can be lowered by lowering the amount of massage content force that can be provided during operation.

One example of spring force at its operating position, provided for illustrative purposes and without limitation, is 520N. Other spring force values may be utilized, as appropriate. One example of spring rate, provided for illustrative purposes and without limitation, is 2.4 N/mm. Other spring rate values may be utilized, as appropriate.

### Damper Sizing

A next component in the chain is then the damper. In embodiments of the robotic massage system described herein, there is a spring that is pushing the arm away (e.g., moving to open the pivoting joint, which would cause the base and the robotic arm to pivot away from the subject). When the magnet is disengaged, the spring force will push the arm away (as the magnetic holding force that was locking the pivoting joint in its closed operating position is now no longer present), until the arm reaches an end stop position. In some embodiments, a safety requirement is specified of a temporal range of time that the robotic arm should take to open and move away from its operating position to a maximum displacement (e.g., maximum pivoted-back position). As one example, such a time range is specified for the robot base to move from its operating position to an opened position in no more than 2 seconds when the robot is in a position where the opening will be fastest, and no more than 15 seconds when the robot is in a position where the opening will be the slowest, such as when fully reaching out in some scenarios.

In some embodiments, the damping rate (in terms of, for example, force per velocity travel from the damper, or N/(m/s)—Newtons per meter per second) is determined based on the spring force, the mass distribution of the robot arm, and the maximum allowed time for the base joint to be fully opened. For example, the damping rate is specified with respect to the spring force and mass distribution of the arm, such that a force-balance equation is closed to result in a certain opening time.

One example of a damper rate, provided for illustrative purposes and without limitation, is 22000 N/(m/s). Other damper rate values may be utilized, as appropriate. As another example, provided for illustrative purposes and without limitation, suppose the spring and the damper travels a stroke of 35 mm between operating position and opened position, and the spring provides 520N force, and the mass of the robot arm in one position contributes to an approximately 200N force at the damper location. Then, in this example, there is a 320N net force pushing the arm away from the user, and causing the arm to accelerate around the pivoting joint until a velocity of the damper is reached where it provides the same 320N net force (the exact value will vary due to non-linearities for the involved components). In this example, with a damping rate of 22000N/(m/s), this will result in a 14.5 mm/s (320N/22000N/(m/s)) velocity, or approximately 2.4 seconds opening time (35 mm/14.5 mm/s). In this example, a relationship between damper rate and opening time is shown.

As described above, in some embodiments, the damping rate and specification of the damper is based on a safety parameter pertaining to maximum allowed opening time during an e-stop event (another example of a safety parameter), as well as the spring force and mass distribution of the arm.

As described above, in some embodiments, a locking component (such as an electromagnet), when released, releases a lock on a multi-position component that is, for example, openable (e.g., pivoting base joint). The openable component then opens from an operating position to an open position. In some embodiments, the openable component is coupled to a base to which a robotic arm is attached. The direction of opening of the openable component is configured such that transitioning from the operating position to the open position causes the robotic arm to move away from an object that the robotic arm interacts with in normal operation. For example, if the robotic arm physically interacts with a human person in the context of providing a robotic massage, then when a triggering event such as an emergency stop event occurs (e.g., because the subject initiated an emergency stop event, power was lost, etc.), the openable component moves from an operating position to an open position, causing the robotic arm to move away from the subject, such that the subject will not be trapped or pinned, and can safely exit or have freedom (e.g., sufficient room) to egress the robotic massage system. Such a system as described herein that creates a separation between a robotic arm and a subject in an emergency stop event is a safety improvement over existing applications of e-stop functionality, which typically freeze a robotic arm in place, which in the context of robotic massage, would risk the subject being trapped by the robotic system.

In some embodiments, the opening motion of the openable component is limited to being in a single direction, where the operating position is one end of the range of motion, and the open position is at the other end of the range of motion. In this way, the arm cannot move closer to the object. In some embodiments, there are no positions in the range of motion of the openable, multi-position component that are associated with the robotic arm being closer to the subject, than when the openable component is in the operating position. For example, the operating position is one end of the range of motion of the openable component, where all other positions in the range of motion of the openable component are associated with the robotic arm (or the base of the robotic arm) being further away from the subject as compared to when the openable component is in the operating position. As one example, the operating position of the openable component is one extreme of the range of motion of the openable component. In some embodiments, the other extreme of the range of motion is a limit of the openable component, or a limit imposed by another component of the system (e.g., an end stop such as end stop pin 1018 of FIG. 10A).

In some embodiments, depending on the mass balance of the system (e.g., center of mass of the arm, spring force, damper specification, etc.), the opening motion of openable component may stop at one of any number of possible end positions/released positions in the range of motion of the openable component. For example, depending on the pose of the robotic arm (which could be in any position when the robotic arm-subject separation mechanism described herein is activated), the openable component may not open all the way to the full extreme of its end position, where it may instead open up and come to rest at an intermediate position that is between the operating position and the full end extreme of the range of motion. The range of motion of the openable component associated with the base (e.g., coupled to or located in the base) facilitates movement of the robotic arm (coupled to the base) away from the subject. That is, starting from an operating position as a starting extreme of the range of motion, the openable component may ultimately open up to, or come to rest at, an end position that is within a range of possible end positions, depending on factors such as the pose of the robotic arm when the emergency stop mechanism is activated, spring force, damping rate, etc.

In some embodiments, components of the robotic safety system described herein are specified (e.g., locking/holding force, spring rate/force, damping rate, etc.) based on a variety of factors and safety requirements/parameters. For example, the spring force is specified based on the possible locations of the center of mass of the robotic arm (which can move over time). For example, the spring force is specified to facilitate opening of the pivoting joint, and reducing (or completely removing) the need for force to be applied by the user to move the robotic arm. The robotic arm may be most difficult to move by a user when the arm is in its maximum extension, as its center of mass will be the furthest away from the pivoting joint. In some embodiments, the spring force (or force that opens the joint or causes it to move from its operating position to an end position) is specified based on the combination of the center of mass of the robotic arm in worst-case scenarios (for the user), such as full arm extension, and a user safety parameter such as the maximum force that can be requested from the user to move the robotic arm away. For example, suppose that massage content force is being applied to a user's back. When the robotic arm is fully extended, and at its maximum reach, then the center of mass of the robotic arm is also at a point that is furthest away from the pivoting joint/lock (which is at the base of the robotic arm), which in turn results in the robotic arm being in its heaviest position for the user to have to push up on (if e-stop were activated when the robotic arm was in this fully extended pose). In some embodiments, the spring is tuned to have sufficient force to push open the pivoting joint when the arm is in its fully extended or maximum reach configuration. This reduces (or removes) user-force needed to move the robotic arm. For example, the spring force value is specified such that regardless of the robotic arm's position when e-stop occurs, little to no force is required or expected to be applied by or exerted from the user to move the end effector, regardless of where the end effector is being applied to the subject's body (e.g., if the user is facing up and can use their hands to move the touchpoint, or if the user is on their back and would use their back to apply the user-force to move the touchpoint and robotic arm away).

As another example, the electromagnetic force (e.g., locking force that holds the pivoting joint locked in its operating position during operation) is based on the massage content force that is translated onto the electromagnet. Multiple dependencies may occur. For example, when the arm is in its maximum extension, this is an arm position that is one of the most difficult for a user to move the robotic arm. On the other hand, when in maximum extension, there is less massage content force translated to the electromagnetic locking mechanism. That is, at full arm extension, there is less force on the locking mechanism due to the translation of massage content force, but the center of mass will also move further away, resulting in different impacts to the spring and the locking mechanism.

In some embodiments, the locking mechanism is tuned or specified with a holding and/or breakaway force. As one example, holding force refers to the amount of force that the locking mechanism can hold with. As one example, the breakaway force refers to an amount of force that will break the lock. The tuning of the breakaway force (the point at which the lock fails and the arm is allowed to break away), provides an additional safety mechanism, passively limiting the maximum massage content force that can be applied to a user during operation. For example, the electromagnet is sized or specified to ensure that a force higher than a certain amount is prevented from being applied to the subject, as a massage content force (that is also translated to the locking mechanism) beyond that threshold would cause the locking mechanism to disengage. That is, the disengagement force for the magnet is set based on a maximum permitted robotic arm force applied to the user by massage content.

The following are further examples of component values. Such values are provided as examples, without limitation. As one example, the locking mechanism, such as an electromagnet, has a force of 1,250 Newtons. As one example, the spring has a spring force of 400 Newtons. As one example, the damper has a damping rate of 22,000 Newtons per meter per second. Various other values may be utilized as appropriate to accommodate other types of robotic arms and configurations (e.g., if the force-balance of the system is changed). Further, the spring may have an initial force at its operating position/compression. As the spring moves, the spring rate may change. Further, the damper may have a non-linear damping rate. The ranges of values for the various components may be designed for a range of operating conditions or a set of target operating conditions. As described above, in some embodiments, the spring force value is determined based on a safety parameter comprising the maximum allowed force that would be required of the user to move the robotic arm away. As described above, in some embodiments, the damping rate value is determined based on a safety parameter comprising a permitted opening/robot-object opening time (also referred to herein as a withdrawal or separation time).

### Further Embodiments of Multi-Position Components

In the above examples, a pivoting joint is an example of a multi-position component that during operation is locked in an operating position (e.g., by the magnetic holding force of a locking electromagnetic mechanism). During an event such as an e-stop event, the lock holding the multi-position component in its operation position is released, such that the multi-position component is now allowed to move/transition to another position. For example, without the holding force locking the multi-position component in place, a force acting on the multi-position component can cause the multi-position component to move to another position in its range of motion. The change in position of the multi-position component away from its operating position is associated with a separation between a robot and an object with which the robot interacts during operation. As one example, the spring force described herein causes an unlocked pivoting joint to open (from its closed operating position). The opening of the pivoting joint, which is coupled to the base of the robot, causes the robotic arm to move away from the table on which a person is resting, creating physical separation between an end effector of the robotic arm and the person.

As described above, as one example, a pivoting joint is one type of multi-position component that can be utilized to facilitate separation between a robot and an object that the robot interacts with. For example, when the electromagnetic locking mechanism that is holding the pivoting joint closed is released, a spring causes the pivoting joint to open (as the holding force counteracting the spring has been removed), which in turn causes the robot arm (and end effector/touch point of the robotic arm) to move away from the object.

The following are alternative embodiments of multi-position components that can be used to facilitate robot-object separation. The following are further embodiments of multi-position components that can transition between various positions. The multi-position components may have a discrete set or continuous range of positions.

In some embodiments, during operation, the multi-position components are locked in an operating position. When a triggering event occurs, such as an e-stop event, the lock is released, where the unlocked multi-position component is movable to another, different position that causes the robot and the object (that the robot interacts with during operation) to be separated or otherwise moved away from each other (where the robot refers to the robotic arm and/or end effector). In the above examples, a pivoting joint is one example of a multi-position component that is utilized (where the pivoting joint can rotate through multiple positions/angles), where there is a single point about which the joint pivots/rotates. The use of the pivoting joint as described herein facilitates relative motion of the arm away from the subject. The use of a rotating or revolute joint such as a pivoting joint is efficient.

Other types of multi-position components, which can be transitioned or moved into various positions (e.g., from operating positions to non-operating positions), may also be utilized to facilitate separation between a robot and an object.

The following are further embodiments of multi-position components that are movable (e.g., openable/closable) between various positions and that are capable of transitioning or moving relatively between operational and non-operational states to facilitate separation between a robotic arm and a subject. Such mechanisms are movable within a range of motion, and are usable to facilitate separation between a robotic arm and an object with which the robotic arm interacts (e.g., during an operating context, such as a robotic massage applied to a person).

### Prismatic Joint/Guiderail

Another example of a multi-position component is one that opens/closes with a linear motion. One example of a multi-position component that can be utilized in embodiments of the robot-object separation system described herein is a prismatic joint on a guiderail. A guiderail is an example of such a sliding component that can be translated between any number of positions. The guiderail can slide in various directions, such as sideways, upwards/downwards, at a slope, etc. As one example, in the operating position, the guiderail is closed. When released (due to the lock releasing), the combined prismatic joint and guiderail can be opened (e.g., via a spring, pneumatics, etc. that is acting with a force to open the guiderail), and a base (with robotic arm attached) attached to the guiderail can then slide or translate away from the subject, creating separation between the robotic arm and the user. The speed of the prismatic joint/guiderail can also be regulated, such as by pneumatics. As another example, a damper can be used to regulate the speed at which the guiderail opens, given a spring force that is acting to move the joint away from its operating position.

FIG. 11 illustrates an embodiment of a robot-object separation system using a prismatic joint with guiderail. In this example, a prismatic joint (that, for example, facilitates linear motion or displacement or translation) is combined on a guide rail (1102) that is attached to the base (e.g., base assembly 1016 that is connected to the massage table). A sliding block (1104) is attached to the robotic arm mounting plate (e.g., a version of mounting plate 1002 to which the robotic arm is mounted).

For example, the block is on a prismatic joint/guide rail combination that allows vertical translation. During operation, the prismatic joint is locked in an operating position corresponding to the block (and thus robotic arm whose base is attached to the block) being at a low position (also referred to in this example as being in a closed operating position). During an e-stop event, the lock on the prismatic joint is released, and the prismatic joint is now openable, and is vertically moveable or translatable along the guiderail. For example, a spring or pneumatics acts to open the prismatic joint to move upwards (away from the ground, for example) along the guiderail to higher, non-operating positions (as compared to the lower, closer to the ground operating position). This opening of the prismatic joint upwards in turn causes the block and robotic arm to move upwards, thereby lifting or raising the robotic arm away from the user.

### Linkages

Another example of a multi-position component that is openable/closable is a four-bar linkage. In this case, rather than the opening of the component being a rotation about a single pivoting point, the opening of the linkage (from an operating position in which the four-bar linkage is in a configuration in which it cannot, for example, be closed further) involves a combination of translation and rotation that causes a base (and the robotic arm to which the base is coupled) attached to the linkage to move away from the subject. For example, the links of the linkage mechanism are configured such that when opening, the links move up and rotate simultaneously.

FIG. 12 illustrates an embodiment of a robot-object separation system using a linkage-based component. In the example of FIG. 12, a linkage implementation is shown to illustrate utilizing a linkage mechanism. In this example, two links (1202 and 1204) are shown. In the example of FIG. 12, an extension of a plate (1002) to which the robotic arm base is mounted is shown at 1206 where a link is attached to the extension to facilitate movement of the robot away from the user. In this example, as the locking magnet releases, there is translation (e.g., of the base plate) downwards simultaneously with the robot (arms) being rotated away from the user, causing the arm to end up in a position further from the user. The specific dimensions and configuration in the pictured linkage of FIG. 12 are provided for purposes of clarity and for illustrative purposes.

### Alternative Embodiments of Robot-Object Separation Systems

In above embodiments, a pivoting joint is opened during a power outage or e-stop event. A locking mechanism is used to lock the pivoting joint during regular operation (when massage content is being applied to a subject). A spring is used that opens the pivoting joint when the locking mechanism is released. The damper regulates the speed at which the pivoting joint is opened (and thus the speed at which the robotic arm falls away from the subject/table). In the above examples, the pivoting base joint is an openable/closable joint that is an example of a multi-position component whose different positions correspond to the robot being different distances away from object. Having an openable joint at the base allows the arms to fall away from the subject during an e-stop event. An arrangement such as that described herein handles or otherwise prevents, for example, the situation where e-stop occurs with the robotic arm in mid-air, preventing the robotic arm from becoming closer to the object (e.g., falling on the user), and ensuring that the robotic arm is only able to move away from the subject.

The following are alternative embodiments of robotic safety systems to facilitate separation between a robotic arm and an object with which the robotic arm interacts during regular operation.

One example alternative robot-object separation system includes an openable/closable multi-position component (e.g., pivoting joint or other displaceable or moveable multi-position component with a range of motion) and a locking mechanism (e.g., electromagnetic lock or other locking mechanism that holds the multi-position component in its operating position, but when the locking mechanism is released, the multi-position component or mechanism is able to transition away from its operating position, facilitating physical separation of the robotic arm and the object), but with fewer other components (e.g., without a spring, without a damper, or without both).

### Damper-Less Displacement System

In above examples, the use of the damper facilitates a number of purposes. One example is safety, where the damper acts as a speed controlling or regulating device that slows the rate at which the arms fall away from the table, preventing the robotic arm from unexpectedly moving and coming in contact with a person or object in the vicinity of the robotic arm, thereby protecting other people. Instead, through the use of a damper and its tuning (taking into account how the reach of the robotic arm changes and affects various dynamics), the robotic arms falls away gracefully. Another benefit provided by the use of the damper is protecting of the robotic arm. For example, the damper protects the robotic arm from being damaged when it hits an end limit (e.g., physical end stop that constrains a joints range of motion and how much the joint can open).

The speed of the displacement of the robotic arm can be regulated in other ways as well. In an alternative embodiment, the pivoting point of the robotic arm and the mass of the robotic arm are moved such that the force-balance of the system does not require a damper to be active when the arms are spaced away from the user. As one example, the pivoting joint is moved further away from the user. This results in the force-balance about that point being much smaller. Further, this would cause the spring force to push the robotic arm away more slowly. The relative spring force, mass distribution of the arm, and the pivoting point may be tuned such that the opening of the pivoting joint proceeds at a slow enough rate that no danger would be imposed.

In another embodiment, a spring is not needed to cause the arm to move away when the locking mechanism is released. For example, suppose that a revolving or pivoting or hinged joint is utilized such as that described herein, that includes a shaft. In some embodiments, repositioning the location of the pivoting joint relative to the center of the mass of the robotic arm (which can vary based on the pose of the robotic arm) facilitates removal of the need for the functionality provided by the spring (to push the arm away from the massage table/subject). For example, moving the pivoting joint to be closer to the massage platform such that the pivoting joint is past the location of the center of mass of the robotic arm when the arm is in its maximum reach, would result in the arm falling away naturally, without requiring the spring.

### Mass-Based Robotic-Object Separation System

In the above examples, a spring is used to force the pivoting joint open when an e-stop event occurs (where the e-stop event causes a locking mechanism to release, either due to user-activation of e-stop, a power outage, etc.). In alternative embodiments, other components are utilized to cause the transitional or multi-position component (e.g., pivoting joint) to move away from its operating position to another position in its range of motion. As one example, gravity is leveraged. For example, in an alternative embodiment to what is shown in the example of FIG. 10A, the spring is removed, and a large mass is instead placed at the base plate. Gravity acting on (e.g., pulling on) the large mass causes a force to be exerted that is trying to open the pivoting joint, and which in normal operation is counteracted by the electromagnetic lock that holds the pivoting joint in its operating position. When the lock is released, gravity acting on the mass causes the mounting plate (and coupled pivoting joint) to move away from its operating position and open, with the robotic arm thus falling away from the platform/table (and create further separation relative to the subject). In some embodiments, the mass placed at the base/mounting plate is determined based on the weight of the robotic arm, its mass distribution in various positions, etc. As one example, the mass is a 20-pound mass.

FIG. 13 illustrates an embodiment of a mass-based displacement system. In this example, a mass 1302 is attached to arm base plate 1002. In such a mass-based separation system, when the electromagnet is released, the mass (which is no longer counteracting the holding strength of the magnet) will pull on the arm base plate 1002, and cause the base plate (with robotic arm) to pivot about the pivoting joint, away from the pivoting joints operation position in its range of motion. As that occurs, the mass is rotating around the shaft of the pivoting joint. When the mass is below the shaft, the mass will no longer be applying rotational torque. The mass and the base plate (with robotic arm) will then come to a stop in a resting position. In some embodiments, the tuning of the amount of mass 1302 applied to the bass plate is used to control the speed at which the mass causes the base plate and robotic arm to pivot, and if slow enough, a damper need not be utilized for speed regulation. In some embodiments, an end stop such as pin 1018 may also not be needed.

### Moving Platform Embodiment

In an emergency stop situation, the robotic arms and the subject should be moved away from each other so that the user can leave the robotic system or so that the user's movement is not otherwise constrained by the robotic arms. In the above examples, separation between the robot and the user is facilitated by moving the robot relative to the user. In some embodiments, this is achieved using the robot base assembly and joint described herein, where the user is on a fixed platform, the base is fixed to the platform, and the robot base assembly is rotated away from the platform (and thus away from the subject on the table). That is, in the above examples, separation between the robot and the object is facilitated by causing the robot (arms) to move away from the subject. In alternative embodiments, the subject is moved away from the arms. As one example, the support structure that supports the object (e.g., platform that the subject is resting on) is moved away from the arms.

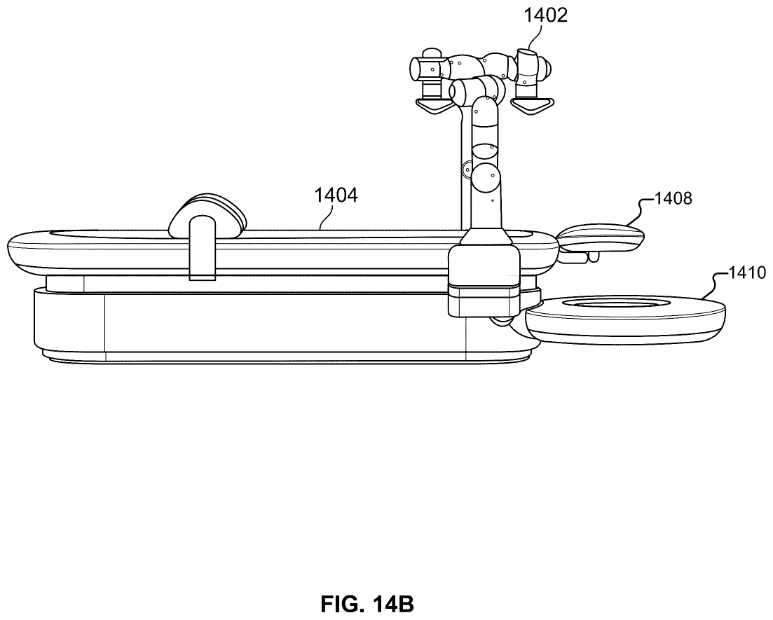

FIGS. 14A and 14B illustrate an embodiment of a robot-object separation system. In the example of FIG. 14A, the robotic massage system is shown with the platform 1404 in a raised, operating position. In this example, the platform (1404) is attached to a moveable mechanism such as prismatic joint 1406 that can move upwards or downwards (e.g., has a range of motion, and can be in one of multiple positions). In the example of FIG. 14A, during operation, the platform is locked in an operating position, such as corresponding to an elevated or raised position of the prismatic joint (where the prismatic joint is locked in such an operating position, for example, by an electromagnetic or mechanical or electromechanical locking mechanism). When the prismatic joint and platform are in the operating position, the robotic arms 1402 are able to interact with a subject on the platform.

When an emergency event occurs (e.g., e-stop is activated or triggered), the lock on the prismatic joint is released, allowing gravity (which is pulling downward on the table, but in operation is counteracted by the holding force of the locking mechanism) to cause the platform 1404 (along with headrest 1408 and armrest 1410) to lower and separate away from the robotic arms (1402). The platform 1404, in its lowered position, is shown in the example of FIG. 14B.

In some embodiments, the arms are mounted such that they do not move with the platform. In this example, the entire platform is on an openable/closable component (component that can be in multiple possible positions). In some embodiments, a damper may also be used to regulate the speed at which the platform lowers.

In the example of FIGS. 14A and 14B, in response to a triggering event such as activation of e-stop, power outage, etc., the platform moves downward, causing the subject to be moved away from the robotic arms. For example, the robotic arms are mounted separately from, or independently of, the platform, so that the platform can move away from the arms.

In the example of FIGS. 14A and 14B, the platform is on a movable component such as a prismatic joint or lifting joint. In some embodiments, during operation (e.g., performing of a massage), the movable component is in an operating position corresponding to the movable/adjustable/openable component being in an open position (e.g., at an extreme in the component's range of motion corresponding to the component such as a joint being open). During operation, the locking mechanism holds the adjustable component (with a range of motion) in its open position (e.g., corresponding to table being at its highest position). During an event such as an e-stop event, release of the lock causes the adjustable component to move or transition from its operating position to an end position that corresponds to the component being more “closed” relative to the operating position, which in turn corresponds to the table being lowered (along with the subject), and a further distance away from the robotic arm (e.g., from the end effectors of the robotic arm).

In some embodiments, the base of the robotic arms is fixed relative to the platform. In this way, the separation between the robot and the user is facilitated by moving the table away from the robotic arms (which do not move). In other embodiments, to facilitate separation, multiple robot-subject relative positioning mechanisms are implemented, where the robotic arms are moved, and the platform is also lowered. For example, both the arms and the subject are moved away from each other. As one example, the platform lowers the user, and the arms are caused to move (e.g., tilt) away from the lowering platform.

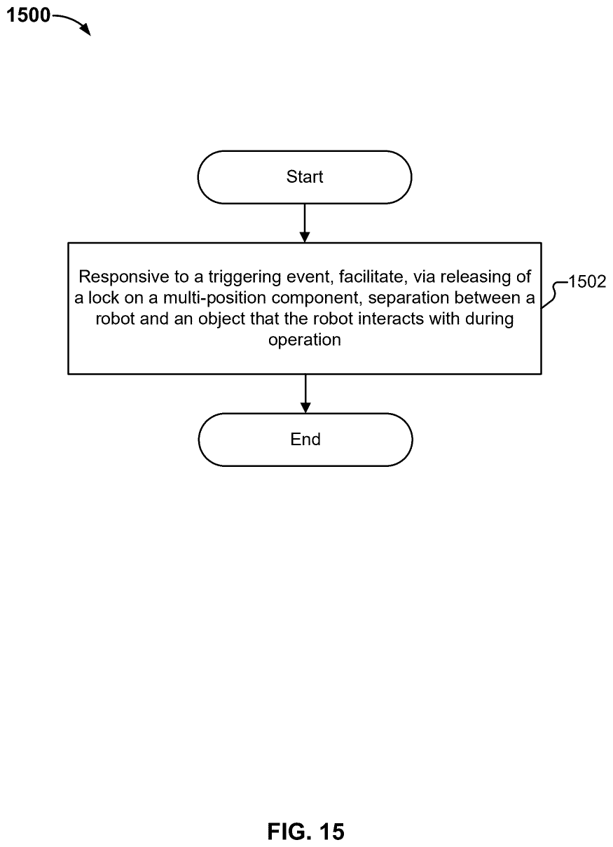

FIG. 15 is a flow diagram illustrating an embodiment of a process for facilitating separation between a robotic arm and an object. In some embodiments, process 1500 is executed by a robotic system such as a robotic massage system as described herein. At 1502, responsive to a triggering event, separation between a robot and an object is facilitated via releasing of a lock on a multi-position component. Releasing of the lock allows the multi-position component to move away from an operating position such that at least one of the robot or the object are moved away from each other.

In some embodiments, during operation, a robot (e.g., robotic arm of the robotic massage system) interacts with the object (e.g., the robotic arm is manipulated to allow an end-effector to interact with the object to perform massage content).

In some embodiments, during operation (e.g., performing of a robotic massage), the multi-position component is locked in an operating position. In various embodiments, multi-position components include openable/closable components. Examples of multi-position components include rotating and/or translating components such as joints (e.g., revolute joints, hinge joints, prismatic joints, etc.), linkages (e.g., four-bar linkages), etc.

In some embodiments, the multi-position component is coupled to the robot. In other embodiments, the multi-position component is coupled to a structure (e.g., platform or table) that supports the object. Multi-position components may be coupled to both the robot and the object-supporting structure to facilitate separation between the robot and the object.

In some embodiments, the triggering event is an indication to cease robot operation (e.g., indication to cease interaction between the robot and the object). In response to the indication to cease robot operation, the lock on the multi-position component in its operating position is released. As one example, an electromagnetic locking mechanism is utilized to lock the multi-position component in its operating position. Mechanical locking mechanism may also be utilized, as appropriate. In some embodiments, during operation, the holding force of the locking mechanism constrains motion or mobility of the multi-position component, locking the multi-position component in an operating position. Responsive to the indication to cease robot operation, the lock is released, permitting movement of the multi-position component via at least one degree of freedom that had been constrained by the holding force of the locking mechanism during operation.

An example of a triggering event is an emergency stop event. An example of an emergency-stop event is user activation or other indication of an emergency-stop being requested. For example, in response to user activation of e-stop, power is removed from an electromagnetic locking mechanism coupled to the multi-position component, disengaging the magnetic holding force that had been holding the multi-position component in its operating position.

Another example of a triggering event is a power outage. The lack of power causes a locking mechanism such as an electromagnet to release its magnetic lock in holding the multi-position component in its operating position.

In some embodiments, movement (e.g., rotation, translation, both, etc.) of the multi-position component away from the operating position causes the robot to move away from the object. For example, the multi-position component is coupled to the robot arm. Moving the multi-position component away from its operating position causes the robot arm to move away from the object. For example, the multi-position component is a pivoting joint. The pivoting joint is coupled to a plate to which the robot arm is mounted. The pivoting joint is part of a base assembly that is mounted to a robotic system. During operation, the pivoting joint is locked in an operating position such that the base of the robotic arm is also fixed, such that the robotic arm can interact with the object via its end effector. When the lock is released, the pivoting joint is now freely moveable (e.g., at least one degree of freedom of movement of the multi-position component is released and made available subsequent to release of the locking mechanism). The pivoting joint can then be pivoted or rotated in a direction corresponding to the robotic arm being tilted away from the object (e.g., tilted away from a structure that supports the object).

In some embodiments, movement of the multi-position component away from the operating position causes the object to move away from the robot. For example, the multi-position component is coupled to a structure, such as a platform, that supports the object that the robot interacts with during operation. For example, a platform is coupled to a prismatic joint. During operation, the prismatic joint is locked in an operating position in which the platform is in a raised position. In response to an indication to cease robotic operation (e.g., cease interaction between the robot and the object), the prismatic joint is released from being locked in the operating position, where at least one degree of freedom of the multi-position component (that had been constrained by the holding force of the locking mechanism) is released (e.g., in the vertical axis, toward the ground) to allow the platform (and the object supported by the platform) to lower, away from the robotic arm(s).

In some embodiments, a force component exerts a force that is directed to moving the multi-position component away from its operating position. One example of such a force component is a spring. In some embodiments, the spring force exerted by the spring is directed to opening a pivoting base joint to which the robotic arm is coupled. During operation, the holding force of the electromagnetic locking mechanism counters the spring force, holding the arm in place. With the holding force removed, the spring force is then able to pivot the robotic arm away from the platform. The spring is an example of an active force component that is actively exerting a force to change the position of the multi-position component (e.g., open a pivoting joint).

In other embodiments, forces such as those exerted by gravity are leveraged to cause the multi-position component to move away from its operating position. For example, in a mass-based system such as that described in conjunction with FIG. 13, a mass is attached to the base plate. The lock holds the pivoting base joint in a fixed operating position. When the lock is released, the placement of the mass, and gravity acting on the attached mass, cause the pivoting base joint to rotate and open, and thus the robot arm to tilt away from the platform.

In some embodiments, a regulating component is used to regulate the speed or velocity at which the multi-position component moves away from its operating position (which in turn regulates the speed at which the robotic arm or support structure coupled to the multi-position component moves). For example, a damper is used to regulate the speed at which a pivoting joint, prismatic joint, linkage, etc. moves away from its operating position.

In some embodiments, the robotic system includes multiple arms. In some embodiments, responsive to an emergency stop event, the separation system described herein is triggered to facilitate separation between the user and the multiple arms. For example, in a system with two arms, both arms are caused to fall or tilt away from the platform that a subject is supported by (e.g., where each arm is associated with a corresponding set of multi-position component and locking mechanism). In some embodiments, each robotic arm is associated with an individual robotic arm safety release mechanism that is individually triggered in response to an e-stop event.

Further details and embodiments of robot-object separation mechanisms and systems are described above. As described above, in various embodiments, facilitating separation between a robot (e.g., robotic arms/end effectors of the robotic arms) and a user (with which the robot interacts during an operational context) includes moving the robot away from the user, moving the user away from the robot (e.g., using embodiments of the movable platform described above), or both. For example, the robotic arms (e.g., the end effector/tool at the end of the robotic arms that interact with the subject) can be moved further away relative to the subject, the subject can be moved further away relative to the robotic arms, and/or both.

The above embodiments of robot-object separation mechanisms for providing physical separation between an object and the end effectors/tools of the robotic arms (that interact with the object during operation) address problems and challenges in having robots interact with subjects such as humans, and are in contrast to typical behavior for existing robotic arms in e-stop scenarios. For example, with existing robotic arms, when an e-stop event is activated or triggered, power is lost, and the robotic arm is locked in position, which in the context of a robotic massage, could result in the user being pinned by the robotic arm. In contrast, using embodiments of the robot-object separation systems described herein, separation between the robot and the object is automatically provided in the event of power outages or e-stop events, such that subjects are able to freely and safely leave the massage table.

For example, in typical robotics or motion systems that have an emergency stop, when the emergency stop is activated, all motion stops. In contrast, the safety systems described herein, when an emergency stop is activated, address a case where a user could be pinned. Using the safety systems described herein, when an event such as activation of emergency stop occurs, some motion does cease, but a degree of freedom is introduced (e.g., in the release of a multi-position component associated with the robot or a platform or other structure that the object is supported by) such that the robot and the object are separated or moved relatively away from each other, avoiding scenarios such as those in which a subject is pinned and unable to extricate themselves from the robot.

The modifications listed herein and other modifications can be made by those in the art without departing from the scope of the disclosure. Although subject matter has been described herein with reference to specific embodiments, the invention(s) is not limited to the above embodiments and the specific configurations shown in the drawings. For example, some components shown can be combined with each other as one embodiment, and/or a component can be divided into several subcomponents, and/or any other known or available component can be added. The processes are not limited to those shown in the examples. Those skilled in the art will appreciate that the invention(s) can be implemented in other ways without departing from the substantive features of the invention. For example, features and embodiments described above can be combined with and without each other. The present embodiments are therefore to be considered in all respects as illustrative and not restrictive. Other embodiments can be utilized and derived therefrom, such that structural and logical substitutions and changes can be made without departing from the scope of this disclosure. This Specification, therefore, is not to be taken in a limiting sense, along with the full range of equivalents to which such claims are entitled.

Such embodiments of the inventive subject matter can be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose can be substituted for the specific embodiments shown. This disclosure is intended to cover any and all adaptations and/or variations of various embodiments. Combinations of the above embodiments, and other embodiments not specifically described herein, will be apparent to those of ordinary skill in the art upon reviewing the above description.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A robotic system, comprising:
a robotic arm that interacts with an object during operation; and
a locking component that, during operation, locks a multi-position component in an operating position;
wherein responsive to a triggering event, locking of the multi-position component in the operating position is released and the multi-position component moves away from its operating position, facilitating separation between the robotic arm and the object.

2. The robotic system of claim 1, further comprising a damper that regulates a speed at which the multi-position component moves away from its operating position.

3. The robotic system of claim 2, wherein the damper is associated with a damper rate that based at least in part on a specified amount of time to move the multi-position component away from its operating position.

4. The robotic system of claim 1, further comprising a spring that causes the multi-position component to move away from its operating position.

5. The robotic system of claim 4, wherein a spring force exerted by the spring is specified based on a maximum permitted force from a user.

6. The robotic system of claim 4, wherein during operation, a holding force of the locking component counters a force of the spring that is directed to moving the multi-position component away from the operating position.

7. The robotic system of claim 1, wherein the multi-position component comprises at least one of a rotating joint, a prismatic joint, or a linkage-based component.

8. The robotic system of claim 1, wherein the locking component comprises an electromagnet.

9. The robotic system of claim 1, wherein a holding force of the locking component is based on a force associated with applying massage content.

10. The robotic system of claim 1, wherein the multi-position component is coupled to the robotic arm.

11. The robotic system of claim 10, wherein the robotic arm is coupled to a base plate, and wherein the multi-position component is coupled to the base plate.

12. The robotic system of claim 11, wherein the multi-position component comprises a pivoting joint located at the base plate.

13. The robotic system of claim 1, wherein the multi-position component is coupled to a support structure that supports the object.

14. The robotic system of claim 13, wherein subsequent to release of the locking on the multi-position component, the support structure moves away from the robotic arm.

15. The robotic system of claim 13, wherein the support structure comprises a platform that supports the object.

16. The robotic system of claim 1, wherein the triggering event comprises user activation of an emergency stop.

17. The robotic system of claim 1, wherein the triggering event comprises a power outage event.

18. The robotic system of claim 1, wherein a range of motion of the multi-position component is limited by an end stop.
